(19) United States
(12) Patent Application Publication
(10) Pub. No.: US 2019/0290726 A1
(43) Pub. Date: Sep. 26, 2019

(54) PROTEIN POLYMER FUSIONS FOR SUBCUTANEOUS DELIVERY OF SMALL MOLECULES

(71) Applicants: John Andrew MACKAY, Los Angeles, CA (US); Jugal P. DHANDHUKIA, Los Angeles, CA (US); Santosh PEDDI, Los Angeles, CA (US); S. Kenny ROBERTS, Los Angeles, CA (US); University of Southern California, Los Angeles, CA (US)

(72) Inventors: John Andrew MACKAY, Los Angeles, CA (US); Jugal P. DHANDHUKIA, Los Angeles, CA (US); Santosh PEDDI, Los Angeles, CA (US); S. Kenny ROBERTS, Los Angeles, CA (US)

(21) Appl. No.: 16/306,825

(22) PCT Filed: Jun. 5, 2017
(86) PCT No.: PCT/US2017/035993

§ 371 Date: Dec. 3, 2018

Related U.S. Application Data
(60) Provisional application No. 62/345,643, filed on Jun. 3, 2016.

(51) Int. Cl. A61K 38/17; A61K 31/436; A61K 9/16; C12N 15/62; A61K 38/18; A61K 47/64

(52) U.S. Cl. CPC A61K 38/1709; A61K 31/436; A61K 47/64; C12N 15/62; A61K 38/1825; A61K 9/16

(57) ABSTRACT

Provided herein are second generation FKBP-ELP drug carriers having physicochemical properties for enhanced systemic circulation and methods for their use.

### CROSS-REFERENCE TO RELATED APPLICATION

This application claims priority under 35 U.S.C. § 119(e) to U.S. Provisional Application No. 62/345,643, filed Jun. 3, 2016, the content of which is hereby incorporated by reference in its entirety.

### TECHNICAL FIELD

This disclosure relates to the subcutaneous delivery of small molecules using genetically engineered protein polymers which consists of ‘cognate’ target receptor of the drug itself.

### BACKGROUND

Systemic delivery of potent and hydrophobic drugs to target cells remains challenging due to the solubility and toxicity profiles associated with such molecules. One such potent drug, rapamycin (Rapa), though clinically approved as an immunosuppressive drug has considerably poor drug like properties. With recent discovery of its anti-proliferative property by inhibiting mTORC1 by forming a complex with cytosolic FK-506 binding protein 12 (FKBP), rapamycin has been studied extensively as viable anti-cancer regimen. However, owing to its extremely low aqueous solubility (˜10 μM) and equally strong potency, systemic administration of rapamycin suffers low bioavailability and causes severe toxic side effects including allergic reactions and cytotoxicity of liver and kidney tissues. Earlier attempts have been made to solubilize rapamycin in organic solvents, however, the in vivo side effects far outweighs the drug solubility profile. Thus, a need in the art exists as to the generation of highly effective drugs with improved toxicity profiles. This disclosure satisfies this need and provides related advantages as well.

### SUMMARY

With aim to improve the bioavailability, solubility and toxicity profile of rapamycin and similar drugs with and without the challenging solubility profile, Applicant demonstrated high avidity rapamycin encapsulation and toxicity-free efficacy by recombinantly fusing its native protein receptor, FKBP to an Elastin-Like Polypeptide (ELP) nanoparticle.

Applicant provides herein an agent comprising, or alternatively consisting essentially of, or yet further consisting of, an elastin-like peptide (ELP) component, a therapeutic agent and an N-terminus ligand and a C-terminus ligand conjugated to the N-terminus and C-terminus of the ELP respectively, wherein each ligand is selected to target a receptor of the therapeutic agent, and wherein the ligands can be the same or different from each other. In one aspect, the molecular weight of the ELP-ligand is between 20 to 150 kDa, or alternatively from about 30 to about 100 kDa, as determined by the method described herein. In one embodiment, the agent further comprises a linker between the ligand and the ELP, which in one aspect, can comprise a thiol reactive linker.

In a further aspect, the therapeutic agent is an anticancer agent or therapeutic. A non-limiting example of the therapeutic agent is rapamycin and the N-terminus and the C-terminus ligands comprise an FK506 binding protein (FKBP), or a biological equivalent thereof, wherein a biological equivalent of the FKBP protein is a peptide that has at least 80% sequence identity to FKBP or a peptide encoded by a polynucleotide that hybridizes under conditions of high stringency to a polynucleotide that encodes FKBP or its complement, wherein conditions of high stringency comprise hybridization reaction at about 60° C. in about 1×SSC and binds rapamycin. In another aspect, the therapeutic agent is cyclosporin A and the ligand comprises cyclophilin A, or a biological equivalent thereof, wherein biological equivalent of cyclophilin A is a peptide that has at least 80% sequence identity to cyclophilin A or a peptide encoded by a polynucleotide that hybridizes under conditions of high stringency to a polynucleotide that encodes cyclophilin A or its complement, wherein conditions of high stringency comprise hybridization reaction at about 60° C. in about 1×SSC and binds cyclosporin A.

In a further aspect, the agent further comprises a detectable label.

In one embodiment, the ELP comprises reference polypeptide (VPGXG)ₙ, (wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about192, and X is an amino acid and may be the same or different and is selected from Ser, Ala, Ile, or Val, or a biological equivalent thereof, wherein a biological equivalent of the ELP is a peptide that has at least 80% sequence identity to the reference polypeptide or a peptide encoded by a polynucleotide that hybridizes under conditions of high stringency to a polynucleotide that encodes the ELP or its complement, wherein conditions of high stringency comprise hybridization reaction at about 60° C. in about 1×SSC.

Further provided is a composition comprising, or alternatively consisting essentially of, or yet further consisting of, the agent as described above and a carrier. In one aspect, the carrier is a pharmaceutically acceptable carrier.

The compositions are useful for delivering a therapeutic agent in vitro, the method comprising, or alternatively consisting essentially of, or yet further consisting of, contacting a tissue with the agent as described herein. Yet further provided is for delivering a drug in vivo, the method comprising, or alternatively consisting essentially of, or yet further consisting of, administering an effective amount of the agent as described above to a subject. Non-limiting examples of subjects include mammals, such as murines, canines, felines, bovines, equines or a human patient. The compositions and methods also can be used for ameliorating the symptoms of a disease or condition or for treating a disease or condition, comprising, or alternatively consisting essentially of, or yet further consisting of, administering an effective amount of the agent as described herein to a subject suffering from the disease or condition or susceptible to the disease or condition. Non-limiting examples of subjects include mammals, such as murines, canines, felines, bovines, equines or a human patient. One can determine if the treatment has been effective by monitoring for clinical or pre-clinical symptoms of the disease or condition. For example, for the treatment of cancer, one can determine if the method reduces tumor growth or metastasis.

Also provided is a kit. The kit comprises an agent or agent as described herein and optionally, instructions for use.

Yet further provided is an isolated polynucleotide encoding an elastin-like peptide (ELP) component that forms a stable nanoparticle above the transition temperature of the ELP and an N-terminus ligand and a C-terminus ligand attached to the N-terminus and C-terminus of the ELP respectively, wherein each ligand is selected to target a receptor of a therapeutic agent, wherein the ELP comprises reference polypeptide (VPGXG)ₙ, (wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about192, and X is an amino acid and may be the same or different and is selected from Ser, Ala, Ile, or Val, or a biological equivalent thereof, wherein a biological equivalent of the ELP is a peptide that has at least 80% sequence identity to the reference polypeptide or a peptide encoded by a polynucleotide that hybridizes under conditions of high stringency to a polynucleotide that encodes the ELP or its complement, wherein conditions of high stringency comprise hybridization reaction at about 60° C. in about 1×SSC.

In one aspect, the N-terminus and/or the C-terminus ligand each comprises polypeptide (FKBP) or a biological equivalent thereof, wherein a biological equivalent of FKBP is a peptide that has at least 80% sequence identity to FKBP or a peptide encoded by a polynucleotide that hybridizes under conditions of high stringency to a polynucleotide that encodes FKBP or its complement, wherein conditions of high stringency comprise hybridization reaction at about 60° C. in about 1×SSC and optionally operatively linked to expression and/or regulatory sequences.

In another aspect, the N-terminus and/or the C-terminus ligand each comprises cyclophilin A or a biological equivalent thereof, wherein a biological equivalent of cyclophilin A is a peptide that has at least 80% sequence identity to cyclophilin A or a peptide encoded by a polynucleotide that hybridizes under conditions of high stringency to a polynucleotide that encodes cyclophilin A or its complement, wherein conditions of high stringency comprise hybridization reaction at about 60° C. in about 1×SSC and optionally operatively linked to expression and/or regulatory sequences.

The isolated polynucleotides can be inserted into a vector, and further comprising regulatory sequences for expression of the polynucleotide. Such vectors can comprise, for example, viral vectors or plasmids for expression in a variety of cells, e.g., prokaryotic or eukaryotic cells, e.g., E. coli, mammalian cells, or yeast cells. The polynucleotides, vectors, or host cells can be combined with each other and/or a carrier, such as a pharmaceutically acceptable carrier.

The vectors and/or host cells can be used for recombinant expression by growing the host cell containing the appropriate polynucleotide under conditions that favor expression of the polypeptide. The recombinant polypeptide can be isolated or purified from the cell or cell culture as appropriate. As is apparent to the skilled artisan, one can chemically synthesize the polynucleotide and/or polypeptide using methods known in the art with the sequence information provided herein.

The ELP-containing polypeptide can be combined with the therapeutic agent under appropriate conditions to encapsulate the agent in the ELP component. Thus, this disclosure also provides a method for preparing the agent by mixing the therapeutic agent and the ELP-fusion and subsequently raising the temperature of the above the transition temperature of the ELP.

### BRIEF DESCRIPTION OF THE DRAWINGS

FIG. 1: Architecture based design and assembly of FKBP-ELP rapamycin carriers. Second generation soluble FKBP-ELP carriers including FV, FA and FAF designed to correlate the effect of polymer circulation and bio-distribution on rapamycin efficacy in comparison to FSI nanoparticles. FAF outperformed other carriers in rapamycin binding, in vitro drug release, in vivo bio-distribution and in vivo efficacy in a mouse tumor xenograft model.

FIGS. 2A-2E: Physicochemical characterization of FKBP-ELP fusion proteins. (FIG. 2A) Copper-stained SDS-PAGE confirmed the identity and purity of all the constructs with single band corresponding to greater than 90% purity. Temperature-concentration phase diagrams of (FIG. 2B) ELPs with and without FKBP and (FIG. 2C) FKBP-ELPs with rapamycin measured optical density at 350 nm by warming the sample mixture solution at the rate of 1° C./min on a UV-Vis spectrophotometer. ELPs phase separate above the indicated lines. (FIG. 2D) Particle size (Rₕ) of all FKBP-ELPs measured with increase in temperature and (FIG. 2E) FKBP-ELPs with rapamycin at 37° C. with respect to time.

FIG. 3: Bivalent FAF shows prolonged in vitro drug release compared to FSI nanoparticles. Release rate of rapamycin from all the FKBP-ELPs was determined by measuring drug concentrations using RP-HPLC from aliquots withdrawn at fixed time intervals while dialyzing formulations under PBS sink conditions. FSI shows a two phase exponential release with a half-lifeᵢₙᵢₜᵢₐₗ of 2 hours and a half-lifeₜₑᵣₘᵢₙₐₗ of ˜60 hours. FV and FA shows a single fast release half-life of ˜15 hours and bivalent FAF shows an apparent half-lifeₜₑᵣₘᵢₙₐₗ of ˜3 months with 80% drug retention after a month of dialysis.

FIGS. 4A-4H: FAF and FSI both suppresses tumor growth in vivo when injected intravenously. Mice with orthotopic implanted breast tumors treated intravenously with (FIG. 4A) PBS (FIG. 4B) FSI-Rapa at 0.25 mg/kg (FIG. 4C) FAF-Rapa at 0.025 mg/kg (FIG. 4D) FAF-Rapa at 0.075 mg/kg and (FIG. 4E) FAF-Rapa at 0.25 mg/kg and (FIG. 4F) FAF-Rapa at 0.75 mg/kg (FIG. 4G) Tumor burden of mice treated with FAF-Rapa at 0.25 mg/kg and 0.75 mg/kg on day 62 were significantly lower compared to PBS than tumor burdens of mice treated with other groups (Tukey's multiple post hoc analysis, α=0.05 with 95% CI). (FIG. 411) No decrease in BW shows no systemic toxicity with any of the Rapa formulations.

FIGS. 5A-5H: FAF outperforms FSI in vivo tumor efficacy model when injected subcutaneously. Mice with orthotopic implanted breast tumors treated subcutaneously with (FIG. 5A) PBS (FIG. 5B) Free Rapa (FIG. 5C) FAF-Rapa (FIG. 5D) FA-Rapa and (FIG. 5E) FSI-Rapa at 0.75 mg/kg drug dose. (FIG. 5F) Tumor burden of mice treated with Free Rapa and FAF-Rapa on day 50 were significantly lower compared to PBS than tumor burdens of mice treated with other groups (Tukey's multiple post hoc analysis, α=0.05 with 95% CI). (FIG. 5G) Kaplan-Meier survival analysis shows Free Rapa and FAF-Rapa had better anti-tumor efficacy than other groups. (FIG. 5H) No decrease in BW shows no systemic toxicity with any of the Rapa formulations.

FIG. 6: Bioluminiscent imaging of FA, FAF and FSI in orthotopic xenograft breast cancer mice. FKBP-ELPs were labeled with near infrared dye Cyanine5.5 and administered subcutaneously in the flank above the hind leg. Mice were anaesthetized and whole body scans were taken using IVIS Spectrum (Perkin Elmer) at 0, 1, 2, 4, 8, 24, and 48 hour post injection. A representative dorsal (D) and ventral (V) scan at 4 and 24 hour is shown from each group of n=4 mice.

FIGS. 7A-7E: Bio-distribution analysis of FA, FAF and FSI in orthotopic xenograft breast cancer mice. Biodistribution of Cy5.5-FA, Cy5.5-FAF and Cy5.5-FSI within (FIG. 7A) Tumor (FIG. 7B) Spleen (FIG. 7C) Liver (FIG. 7D) Kidney and (FIG. 7E) Injection site was analyzed by measuring fluorescence quantified by drawing region of interest (ROIs) on all the major organs and plotted against time. Values indicate mean±SD with n=4 mice per group.

FIGS. 8A-8B: FA and FAF shows high accumulation in tumor and clearance organs compared to FSI. After 48 hours, mice were euthanized and organs were scanned for fluorescence. (FIG. 8A) A representative mouse is shown from each group of n=4. (FIG. 8B) The fluorescence intensity was quantified by drawing ROIs on all the organs. Values indicate mean±SD.

### DETAILED DESCRIPTION

### Definitions

The practice of the present disclosure will employ, unless otherwise indicated, conventional techniques of tissue culture, immunology, molecular biology, microbiology, cell biology and recombinant DNA, which are within the skill of the art. See, e.g., Sambrook et al., (1989) Molecular Cloning: A Laboratory Manual, 2nd edition; Ausubel et al., eds. (1987) Current Protocols In Molecular Biology; MacPherson, B. D. Hames and G. R. Taylor eds., (1995) PCR 2: A Practical Approach; Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this disclosure.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. In the context of this application, the active agent is the ELP-containing a ligand and therapeutic agent as described herein. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this disclosure, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

The term “pharmaceutically acceptable carrier” (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

As used herein, the term “patient” or “subject” intends an animal, a mammal or yet further a human patient. For the purpose of illustration only, a mammal includes but is not limited to a human, a feline, a canine, a simian, a murine, a bovine, an equine, a porcine or an ovine.

The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

The term “therapeutic” refers to an agent or component capable of inducing a biological effect in vivo and/or in vitro. The biological effect may be useful for treating and/or preventing a condition, disorder, or disease in a subject or patient. A therapeutic may include, without limitation, a small molecule, a nucleic acid, or a polypeptide. Non-limiting examples of such include rapamycin and cyclosporin A.

As used herein, the term “elastin-like peptide (ELP) component” intends a polypeptide that forms stable nanoparticle (also known as a micelle) above the transition temperature of the ELP. In one aspect, the ELP component comprises, or alternatively consists essentially of, or yet further consists of the polypeptide (VPGXG)ₙ, wherein X is any amino acid, or alternatively Ala, Ser, Ile or Val, and wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about192. In one aspect the ELP is S48I48 having the sequence G(VPGSG)ₙ(VPGIG)ₙY, (wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about 192, wherein in one aspect, S48I48 comprises, or alternatively consists essentially of, or yet further consists of the amino acid sequence G(VPGSG)₄₈(VPGIG)₄₈Y, or a biological equivalent thereof. A biological equivalent of an ELP polypeptide is a peptide that has at least 80% sequence identity to the reference polypeptide or a peptide encoded by a polynucleotide that hybridizes under conditions of high stringency to a polynucleotide that encodes ELP polypeptide or its complement, wherein conditions of high stringency comprise hybridization reaction at about 60° C. in about 1×SSC. In one aspect, the biological equivalent will retain the characteristic or function of forming a nanoparticle (also known as a micelle) when the biological equivalent is raised above the transition temperature of the biological equivalent or, for example, the transition temperature of S48I48.

Rapamycin is a small molecule drug with the IUPAC name (3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29(4H,6H,31H)-pentoneis). It is an immunosuppressant drug used to prevent rejection in organ transplantation and has been used in the treatment of cancers. It is marketed under the trade name Rapamune™ by Pfizer.

The mammalian target of rapamycin is known as mTOR or FK506 binding protein 12-rapamycin associated protein 1 (FRAP1, referenced herein is FK506 Binding Protein or “FKBP”), is a protein that in humans is encoded by the FRAP1 gene. The protein and gene sequence encoding the protein are disclosed under GenBank Accession No. NG_033239 (last accessed on Sep. 6, 2013). mTOR is a serine/threonine protein kinase that regulates cell growth, proliferation, cell survival, protein synthesis among other functions.

Cyclosporin A is a small molecule immunosuppressant drug widely used in organ transplants and has been successfully used in the treatment of cardiac disease. The IUPAC name for the drug is (3S,6S,9S,12R,15S,18S,21S,24S,30S,33S)-30-Ethyl-33-[(1R,2R,4E)-1-hydroxy-2-methyl-4-hexen-1-yl]-6,9,18,24-tetraisobutyl-3,21-diisopropyl-1,4,7,10,12,15,19,25,28-nonamethyl-1,4,7,10,13,16,19,22,25,28,31-undecaazacyclotritriacontane-2,5,8,11,14,17,20,23,26,29,32-undecone. It is sold under the trade names Neoral™ or Sandimmune™. It binds the cytosolic protein cyclosporine A.

Cyclophilin A is also known as peptidylprolyl isomerase A. It is found in the cytosol. The sequence of the human protein and polynucleotide encoding the protein is disclosed under GenBank Accession No.: NP_066953 (last accessed on Oct. 7, 2013). A published amino acid sequence comprises mvnptvffdi avdgeplgry sfelfadkvp ktaenfrals tgekgfgykg scfhriipgf mcqggdftrh ngtggksiyg ekfedenfil khtgpgilsm anagpntngs qffictakte wldgkhvvfg kvkegmnive amerfgsrng ktskkitiad cgqle.

Everolimus is the 40-O-(2-hydroxyethyl) derivative of sirolimus and works similarly to sirolimus as an inhibitor of the mammalian target of rapaycin. It is marketed under the tradenames Zortress (USA) and Certican (Europe and other countries) in transplantation medicine, and Afinitor in oncology. Everolimus also is available with Biocon with the brand name of Evertor. It is used as an immunosuppresent to prevent rejection of organ transplants and the treatment of tumors such as renal cell cancer. The compound also is known as dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0 hexatriaconta-16,24,26,28-tetraene-2,3,10,14,20-pentone.

Temsirolimus is (CCI-779) is a derivative of sirolimus and is sold as Torisel. It is an intravenous drug for the treatment of renal cell carcinoma, developed by Wyeth Pharmaceuticals. It also is approved by the European Medicines Agency (EMEA) on November 2007. The compound also is known as (1R,2R,4S)-4-{(2R)-2-[(3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate.

Ridaforolimus (also known as AP23573 and MK-8669; formerly known as Deforolimus) is an investigational targeted and small-molecule inhibitor of the protein mTOR. The compound also is known as (1R,2R,4S)-4-[(2R)-2-[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate.

Tacrolimus (also FK-506 or fujimycin, trade names Prograf, Advagraf, Protopic) is an immunosuppressive drug that is mainly used after allogeneic organ transplant to reduce patient rejection. The drug also is known as 3S[3R*[E(1S*,3S*,4S*)],4S*,5R*,8S*,9E,12R*,14R*,15S*,16R*,18S*,19S*,26aR*5,6,8,11,12,13, 14,15,16,17,18,19,24,25,26,26a-hexadecahydro-5,19-dihydroxy-3-[2-(4-hydroxy-3 methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone, monohydrate.

As used herein, the term “biological equivalent thereof” is used synonymously with “equivalent” unless otherwise specifically intended. When referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 60%, or 65%, or 70%, or 75%, or 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. Alternatively, a biological equivalent is a peptide encoded by a nucleic acid that hybridizes under stringent conditions to a nucleic acid or complement that encodes the peptide or with respect to polynucleotides, those hybridize under stringent conditions to the reference polynucleotide or its complement. Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

A polynucleotide or polynucleotide region (or a polypeptide or polypeptide region) having a certain percentage (for example, about 60%, 65%, 70%, 75%, 80%, 85%, 90%, 95% or 97%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

An “equivalent” of a polynucleotide or polypeptide refers to a polynucleotide or a polypeptide having a substantial homology or identity to the reference polynucleotide or polypeptide. In one aspect, a “substantial homology” is greater than about 60%, 65%, 70%, 75%, 80%, 85%, 90%, 95% or 98% homology.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Regulatory polynucleotide sequences” intends any one or more of promoters, operons, enhancers, as known to those skilled in the art to facilitate and enhance expression of polynucleotides.

An “expression vehicle” is a vehicle or a vector, non-limiting examples of which include viral vectors or plasmids, that assist with or facilitate expression of a gene or polynucleotide that has been inserted into the vehicle or vector.

A “delivery vehicle” is a vehicle or a vector that assists with the delivery of an exogenous polynucleotide into a target cell. The delivery vehicle may assist with expression or it may not, such as traditional calcium phosphate transfection compositions.

“An effective amount” refers to the amount of an active agent or a pharmaceutical composition sufficient to induce a desired biological and/or therapeutic result. That result can be alleviation of the signs, symptoms, or causes of a disease, or any other desired alteration of a biological system. The effective amount will vary depending upon the health condition or disease stage of the subject being treated, timing of administration, the manner of administration and the like, all of which can be determined readily by one of ordinary skill in the art.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

As used herein, to “treat” further includes systemic amelioration of the symptoms associated with the pathology and/or a delay in onset of symptoms. Clinical and sub-clinical evidence of “treatment” will vary with the pathology, the subject and the treatment.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, topical application, intraperitoneal, intravenous, subcutaneous and by inhalation. An agent of the present disclosure can be administered for therapy by any suitable route of administration. It will also be appreciated that the preferred route will vary with the condition and age of the recipient, and the disease being treated.

The agents and compositions of the present disclosure can be used in the manufacture of medicaments and for the treatment of humans and other animals by administration in accordance with conventional procedures, such as an active ingredient in pharmaceutical compositions.

As used herein, the term “patient” or “subject” intends an animal, a mammal or yet further a human patient. For the purpose of illustration only, a mammal includes but is not limited to a human, a feline, a canine, a simian, a murine, a bovine, an equine, a porcine or an ovine. In terms of cells, the term “mammalian cells” includes, but is not limited to cells of the following origin: a human, a feline, a canine, a simian, a murine, a bovine, an equine, a porcine or an ovine.

As used herein, the term “detectable label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescent labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

### Modes For Carrying Out The Disclosure

ELPs are genetically encodable protein polymers with amino acid sequence (VPGXG)ₙ where X represents a guest residue and n represents the number of pentameric repeats. ELPs demonstrate reversible phase separation property which makes them water soluble at lower temperatures but assemble or phase separate above a critical inverse transition temperature (Tₜ). They make attractive scaffolds for in vivo applications because they are biodegradable, can be genetically fused with different protein domains, peptides or therapeutics, and can be expressed and purified from prokaryotic systems without the need of chemical synthesis. FKBP, which is the native cytosolic receptor for rapamycin, was attached genetically to an ELP diblock copolymer comprised of a hydrophilic ELP at the N terminus with X=Ser and n=48 repeats followed by a hydrophobic ELP with X=Ile and n=48 repeats (S48I48). FKBP-S48I48 (FSI) was expressed and purified from E. coli as rapamycin drug carrier and was evaluated for rapamycin efficacy through in vitro MTS assay and in vivo tumor xenograft model with human MDA-MB-468 breast cancer cells. MDA-MB-468 are triple negative cells lacking estrogen, progesterone and HER2+ receptors. In addition, these cells are devoid of PTEN phosphatase which results in extensive phosphorylation of AKT kinase and a downstream active mTORC1. Due to these cellular genotypic features, MDA-MB-468 remains one of the difficult tumors to treat. Rapamycin loaded with FSI not only improved the solubility of the drug but also the blood circulation half-life. FSI-rapamycin formulation was efficacious in completely suppressing tumor growth both in vitro and in vivo without any peripheral tissue toxicity compared to free drug which showed significant side effects.

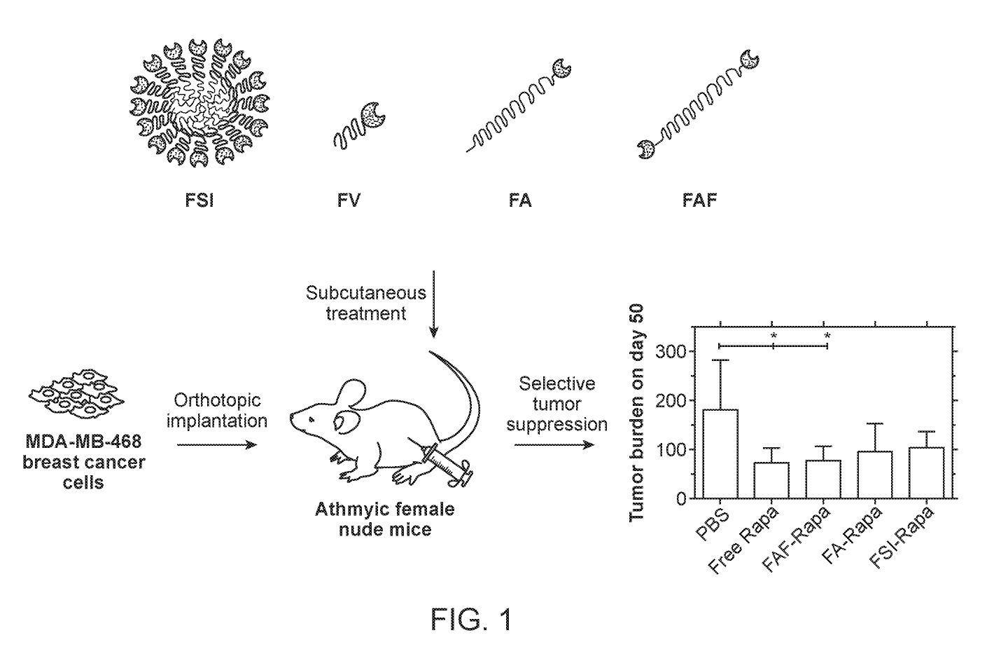

Although efficacious in vivo, what has been lacking is an in-depth understanding of how the ELP architecture influences the carrier circulation, bio-distribution and in vivo efficacy of rapamycin. To study these parameters, Applicants designed and synthesized second generation FKBP-ELP carriers with different molecular weight and particle size to correlate the effect of polymer circulation and bio-distribution on rapamycin efficacy. The second generation FKBP-ELPs were synthesized having ELP backbone with X=Val or Ala and n=48 or 192 repeats, represented as FKBP-V48 (FV) and FKBP-A192 (FA). To compare the effect of FKBP valency on rapamycin binding and release, a bivalent drug carrier with orientation FKBP-A192-FKBP (FAF) was synthesized by attaching FKBP domain at both the N-and C-terminus of A192 ELP. These three carriers have ELP sequence with single guest residue, in other words, they do not assemble into nanoparticles and remain soluble at physiological temperatures. The nomenclature, amino acid sequence and physicochemical properties of all FKBP-ELPs evaluated in this study are shown in Table 1. These carriers were compared with FSI nanoparticles for rapamycin binding, drug release, in vivo bio-distribution and as rapamycin carriers in vivo in a mouse tumor xenograft model. Applicants discovered that among the second generation carriers, FAF outperformed other FKBP-ELPs with extensive drug release half-life, enhanced stability, higher in vivo tumor distribution and enhanced in vivo efficacy in a xenograft tumor mouse model (FIG. 1).

Applicant reported one such nanomedicine application of recombinant fusion of FK-506 binding protein 12 (FKBP) to ELP nanoparticles (FSI) as rapamycin (Rapa) carriers to triple negative breast cancer cells. FSI nanoparticles loaded with rapamycin assembles into Rₕ ˜25 nm particles at 37° C. and suppresses tumor growth in vivo with significantly low toxicity compared to free drug. In this disclosure, Applicant describes the influence of ELP architecture on the structure-dependence efficacy of different FKBP-ELP formulations as rapamycin carriers. Applicant synthesized and characterized second generation FKBP-ELP drug carriers having physicochemical properties for enhanced systemic circulation. These second generation carriers were synthesized (i) without nanoparticle assembly property having soluble linear ELP backbones, (ii) with molecular weights from 30-100 kDa range, (iii) particle size above renal filtration cutoff and (iv) with 1 or 2 copies of FKBP protein per ELP for improved drug binding and release. These carriers were compared with previously reported FSI nanoparticles for drug release, drug binding, in vivo bio-distribution and efficacy in a MDA-MB-468 human xenograft mouse model via intravenous (IV) and subcutaneous (SC) routes of administration.

With these concepts in mind, the following embodiments are provided.

### Elastin-Like Polypeptides (ELPs)

Elastin-like-polypeptides (ELPs) are a genetically engineered polypeptide with unique phase behavior (see for e.g. S. R. MacEwan, et al., Biopolymers 94(1) (2010) 60-77), which promotes recombinant expression, protein purification, and self-assembly of nanostructures (see for e.g. A. Chilkoti, et al., Advanced Drug Delivery Reviews 54 (2002) 1093-1111). ELPs are artificial polypeptides composed of repeated pentapeptide sequences, (Val-Pro-Gly-Xaa-Gly)ₙ derived from human tropoelastin, where Xaa is the “guest residue” which is any amino acid, an amino acid analog or amino acid derivative thereof. In one embodiment, Xaa is any amino acid except proline. In another aspect, the guest residue is any one of Ile, Val, Ala or Ser This peptide motif displays rapid and reversible de-mixing from aqueous solutions above a transition temperature, Tₜ. Below Tₜ, ELPs adopt a highly water soluble random coil conformation; however, above Tₜ, they separate from solution, coalescing into a second aqueous phase. The Tₜ of ELPs can be tuned by choosing the guest residue and ELP chain length as well as fusion peptides at the design level (see for e.g. MacEwan S R, et al., Biopolymers 94(1): 60-77). The ELP phase is both biocompatible and highly specific for ELPs or ELP fusion proteins, even in complex biological mixtures. Genetically engineered ELPs are monodisperse, biodegradable, non-toxic. Throughout this description, ELPs are identified by the single letter amino acid code of the guest residue followed by the number of repeat units, n. For example, S48I48 represents a diblock copolymer ELP with 48 serine (S) pentamers at the amino terminus and 48 isoleucine (I) pentamers at the carboxy terminus.

Described herein are ELP fusion proteins, which can be self-assembled into nanoparticles (alternatively known as micelles). The diameter of the nanoparticle can be from about 1 to about 1000 nm or from about 1 to about 500 nm, or from about 1 to about 100 nm, or from about 1 to about 50 nm, or from about 20 to about 50 nm, or from about 30 to about 50 nm, or from about 35 to about 45 nm, or from about 4 to about 30 nm, or from about 8 to about 30 nm, or about 9 to 25 nm. In one embodiment, the diameter is about 40 nm. These nanoparticles can be high efficiently internalized, e.g. into lacrimal gland acinar cell (LGAC). The fusion proteins are composed of elastin-like-polypeptides and high affinity polypeptides. These fusion proteins can be expressed from a variety of expression systems known to those skilled in the art and easily purified by the phase transition behavior of ELPs. These ELP fusion proteins are able to conjugate small molecules, such as, for example, chemotherapeutic agents, anti-inflammation agents, antibiotics and polypeptides and other water soluble drugs. In addition, the ELP nanoparticles are useful for carrying DNA, RNA, protein and peptide-based therapeutics.

ELPs have potential advantages over chemically synthesized polymers as drug delivery agents. First, because they are biosynthesized from a genetically encoded template, ELPs can be made with precise molecular weight. Chemical synthesis of long linear polymers does not typically produce an exact length, but instead a range of lengths. Consequently, fractions containing both small and large polymers yield mixed pharmacokinetics and biodistribution. Second, ELP biosynthesis produces very complex amino acid sequences with nearly perfect reproducibility. This enables very precise selection of the location of drug attachment. Thus drug can be selectively placed on the corona, buried in the core, or dispersed equally throughout the polymer. Third, ELP can self-assemble into multivalent nanoparticles that can have excellent site-specific accumulation and drug carrying properties. Fourth, because ELP are designed from native amino acid sequences found extensively in the human body they are biodegradable, biocompatible, and tolerated by the immune system. Fifth, ELPs undergo an inverse phase transition temperature, Tₜ, above which they phase separate into large aggregates. By localized heating, additional ELP can be drawn into the target site, which may be beneficial for increasing drug concentrations.

As disclosed herein, the ELPs of this disclosure are attached to a receptor ligand that binds to therapeutic small-molecule agents. Non-limiting examples of such include FKBP that is the ligand for rapamycin or cyclophilin which is the ligand for cyclosporin A. The ELP and receptor are fused directly through a covalent peptide linkage, which is genetically encoded at the level of the DNA.

A therapeutic such as a drug, for example, may be attached to the ELP through cysteine, lysine, glutamic acid or aspartic acid residues present in the polymer. In some embodiments, the cysteine, lysine, glutamic acid or aspartic acid residues are generally present throughout the length of the polymer. In some embodiments, the cysteine, lysine, glutamic acid or aspartic acid residues are clustered at the end of the polymer. In some embodiments of the presently described subject matter, therapeutics are attached to the cysteine residues of the ELP using thiol reactive linkers. In some embodiments of the presently described subject matter, therapeutics are attached to the lysine residues of the high molecular weight polymer sequence using NHS (N-hydroxysuccinimide) chemistry to modify the primary amine group present on these residues. In some embodiments of the presently described subject matter, therapeutics are attached to the glutamic acid or aspartic acid residues of the ELP using EDC (1-Ethyl-3-[3-dimethylaminopropyl]carbodiimide Hydrochloride) chemistry to modify the carboxylic acid group present on the ELP residues.

The therapeutic associated with the ELP may be hydrophobic or hydrophilic. Which the drug is hydrophobic, attachment to the terminus of the ELP may facilitate formation of the multivalent nanoparticle. The number of drug particles attached to the ELP can be from about 1 to about 30, or from about 1 to about 10, or about 1, 2, 3, 4, 5, 6, 7, 8, 9, or 10. In some embodiments, the attachment points for a therapeutic are equally distributed along the backbone of the ELP, and the resulting drug-ELP is prevented from forming nanoparticle structures under physiological salt and temperature conditions.

In addition to therapeutics, the ELPs may also be associated with a detectable label that allows for the visual detection of in vivo uptake of the ELPs. Suitable labels include, for example, fluorescein, rhodamine, tetramethylrhodamine, eosin, erythrosin, coumarin, methyl-coumarins, pyrene, Malacite green, Alexa-Fluor®, stilbene, Lucifer Yellow, Cascade Blue™, and Texas Red. Other suitable optical dyes are described in Haugland, Richard P. (1996) Molecular Probes Handbook.

In certain embodiments, the ELP components include polymeric or oligomeric repeats of the pentapeptide (VPGXG)ₙ, wherein n is an integer representing the number of repeats between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about192, and wherein the guest residue X (also denoted as Xaa herein) is any amino acid, that in one aspect, excludes proline. X may be a naturally occurring or non-naturally occurring amino acid. In some embodiments, X is selected from alanine, arginine, asparagine, aspartic acid, cysteine, glutamic acid, glutamine, glycine, histidine, isoleucine, leucine, lysine, methionine, phenylalanine, serine, threonine, tryptophan, tyrosine and valine. In some embodiments, X is a natural amino acid other than proline or cysteine. In one aspect, it is Ala, Val, Ser, or Ile.

The guest residue X may be a non-classical (non-genetically encoded) amino acid. Examples of non-classical amino acids include: D-isomers of the common amino acids, 2,4-diaminobutyric acid, α-amino isobutyric acid, A-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs in general.

Selection of X is independent in each ELP structural unit (e.g., for each structural unit defined herein having a guest residue X). For example, X may be independently selected for each structural unit as an amino acid having a positively charged side chain, an amino acid having a negatively charged side chain, or an amino acid having a neutral side chain, including in some embodiments, a hydrophobic side chain.

In each embodiment, the structural units, or in some cases polymeric or oligomeric repeats, of the ELP sequences may be separated by one or more amino acid residues that do not eliminate the overall effect of the molecule, that is, in imparting certain improvements to the therapeutic component as described. In certain embodiments, such one or more amino acids also do not eliminate or substantially affect the phase transition properties of the ELP component (relative to the deletion of such one or more amino acids).

The ELP component in some embodiments is selected or designed to provide a Tₜ ranging from about 10 to about 80° C., such as from about 35 to about 60° C., or from about 38 to about 45° C. In some embodiments, the Tₜ is greater than about 40° C. or greater than about 42° C., or greater than about 45° C., or greater than about 50° C. The transition temperature, in some embodiments, is above the body temperature of the subject or patient (e.g., >37° C.) thereby remaining soluble in vivo, or in other embodiments, the Tₜ is below the body temperature (e.g., <37° C.) to provide alternative advantages, such as in vivo formation of a drug depot for sustained release of the therapeutic agent.

The Tₜ of the ELP component can be modified by varying ELP chain length, as the Tₜ generally increases with decreasing MW. For polypeptides having a molecular weight >100,000, the hydrophobicity scale developed by Urry et al. (PCT/US96/05186, which is hereby incorporated by reference in its entirety) is preferred for predicting the approximate Tₜ of a specific ELP sequence. However, in some embodiments, ELP component length can be kept relatively small, while maintaining a target Tₜ, by incorporating a larger fraction of hydrophobic guest residues (e.g., amino acid residues having hydrophobic side chains) in the ELP sequence. For polypeptides having a molecular weight <100,000, the Tₜ may be predicted or determined by the following quadratic function: Tₜ=M₀+M₁X+M₂X² where X is the MW of the fusion protein, and M₀=116.21; M₁=−1.7499; M₂=0.010349.

While the Tₜ of the ELP component, and therefore of the ELP component coupled to a therapeutic component, is affected by the identity and hydrophobicity of the guest residue, X, additional properties of the molecule may also be affected. Such properties include, but are not limited to solubility, bioavailability, persistence, and half-life of the molecule.

### Expression of Recombinant Proteins

ELPs and other recombinant proteins described herein can be prepared by expressing polynucleotides encoding the polypeptide sequences of this disclosure in an appropriate host cell, i.e., a prokaryotic or eukaryotic host cell. This can be accomplished by methods of recombinant DNA technology known to those skilled in the art. It is known to those skilled in the art that modifications can be made to any peptide to provide it with altered properties. Polypeptides of the disclosure can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that beta-turn spiral secondary structure or random secondary structure is preferred.

The ELPs can be expressed and purified from a suitable host cell system. Suitable host cells include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escherichia coli, Salmonella enterica and Streptococcus gordonii. In one embodiment, the host cell is E. coli. The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells includes Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

### Protein Purification

The phase transition behavior of the ELPs allows for easy purification. The ELPs may also be purified from host cells using methods known to those skilled in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, exclusion chromatography, polyacrylamide gel electrophoresis, affinity chromatography, or isoelectric focusing. A particularly efficient method of purifying peptides is fast protein liquid chromatography or even HPLC. In the case of ELP compositions protein purification may also be aided by the thermal transition properties of the ELP domain as described in U.S. Pat. No. 6,852,834.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “[n]-fold purification number” wherein “n” is an integer. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxyapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

### Pharmaceutical Compositions

Pharmaceutical compositions are further provided. The compositions comprise a carrier and an agent, an ELP-fusion with a ligand, or a polynucleotide encoding the ELP-fusion, as described herein or other compositions (e.g., polynucleotide, vector system, host cell) as described herein. The carriers can be one or more of a solid support or a pharmaceutically acceptable carrier. In one aspect, the compositions are formulated with one or more pharmaceutically acceptable excipients, diluents, carriers and/or adjuvants. In addition, embodiments of the compositions include ELPs, formulated with one or more pharmaceutically acceptable auxiliary substances.

The disclosure provides pharmaceutical formulations in which the one or more of an agent, ELP-fusion with a ligand, or a polynucleotide, vector or host cells can be formulated into preparations for injection or other appropriate route of administration in accordance with the disclosure by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents.

Aerosol formulations provided by the disclosure can be administered via inhalation. For example, embodiments of the pharmaceutical formulations of the disclosure comprise a compound of the disclosure formulated into pressurized acceptable propellants such as dichlorodifluoromethane, propane, nitrogen and the like.

Embodiments of the pharmaceutical formulations of the disclosure include those in which the composition is formulated in an injectable composition. Injectable pharmaceutical formulations of the disclosure are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the disclosure.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Routes of administration applicable to the methods and compositions described herein include intranasal, intraperitoneal, intramuscular, subcutaneous, intradermal, topical application, intravenous, nasal, oral, inhalation, intralacrimal, retrolacrimal perfusion along the duct, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes. In one embodiment, the composition comprising the ELP and agent is administered intralacrimally through injection. In further embodiments, the composition is administered systemically, topically on top of the eye, by retrolacrimal perfusion, or intranasally.

### Treatment of Disease

In one aspect, this disclosure provides methods and compositions useful in treating cancer, e.g., breast cancer. As is apparent to those of skill in the art, the cancer to be treated will vary with the therapeutic agent encapsulated and the ligand of the ELP. Non-limiting examples of additional disorders can include, age-related macular degeneration, Sjögren's syndrome, autoimmune exocrinopathy, diabetic retinopathy, graft versus host disease (exocrinopathy associated with) retinal venous occlusions, retinal arterial occlusion, macular edema, postoperative inflammation, uveitis retinitis, proliferative vitreoretinopathy and glaucoma. In one embodiment, the disease is Sjögren's syndrome. In another embodiment, the disease is keratoconjunctivitis sicca (dry eye). In another embodiment the disease is scleritis. In another embodiment the disease is glaucoma.

### Use of Compounds for Preparing Medicaments

The ELPs of the present disclosure are also useful in the preparation of medicaments to treat a variety of pathologies as described herein. The methods and techniques for preparing medicaments of a composition are known in the art. For the purpose of illustration only, pharmaceutical formulations and routes of delivery are detailed herein. In one aspect when the ELP is combined with another therapy or therapeutic agent, provided herein the compositions are useful in the preparation of combination compositions that can be simultaneously or concurrently administered.

Thus, one of skill in the art would readily appreciate that any one or more of the compositions described above, including the many specific embodiments, can be used by applying standard pharmaceutical manufacturing procedures to prepare medicaments to treat the many disorders described herein. Such medicaments can be delivered to the subject by using delivery methods known in the pharmaceutical arts.

### Kits

The ELPs as described herein, can be provided in kits. The kits can further contain additional therapeutics and optionally, instructions for making or using the ELPs. In a further aspect, the kit contains reagents and instructions to perform a screen as detailed herein.

### Screening Assays

This disclosure also provides screening assays to identify potential therapeutic agents of known and new compounds and combinations. For example, one of skill in the art can also determine if the ELP provides a therapeutic benefit in vitro by contacting the ELP or combination comprising the ELP with a sample cell or tissue to be treated. The cell or tissue can be from any species, e.g., simian, canine, bovine, ovine, rat, mouse or human.

The contacting can also be performed in vivo in an appropriate animal model or human patient. When performed in vitro, the ELPs can be directly added to the cell culture medium. When practiced in vitro, the method can be used to screen for novel combination therapies, formulations or treatment regimens, prior to administration to an animal or a human patient.

In another aspect, the assay requires contacting a first sample comprising suitable cells or tissue (“control sample”) with an effective amount of an ELP as disclosed herein and contacting a second sample of the suitable cells or tissue (“test sample”) with the ELP, agent or combination to be assayed. In one aspect in the case of cancer, the inhibition of growth of the first and second cell samples are determined. If the inhibition of growth of the second sample is substantially the same or greater than the first sample, then the agent is a potential drug for therapy. In one aspect, substantially the same or greater inhibition of growth of the cells is a difference of less than about 1%, or alternatively less than about 5% or alternatively less than about 10% , or alternatively greater than about 10% , or alternatively greater than about 20%, or alternatively greater than about 50%, or alternatively greater than about 90%. The contacting can be in vitro or in vivo. Means for determining the inhibition of growth of the cells are well known in the art.

In a further aspect, the test agent is contacted with a third sample of cells or tissue comprising normal counterpart cells or tissue to the control and test samples and selecting agents that treat the second sample of cells or tissue but does not adversely affect the third sample. For the purpose of the assays described herein, a suitable cell or tissue is described herein such as cancer or other diseases as described herein. Examples of such include, but are not limited to cancer cell or tissue obtained by biopsy, blood, breast cells, colon cells.

Efficacy of the test composition is determined using methods known in the art which include, but are not limited to cell viability assays or apoptosis evaluation.

In yet a further aspect, the assay requires at least two cell types, the first being a suitable control cell.

The assays also are useful to predict whether a subject will be suitably treated by this disclosure by delivering an ELP to a sample containing the cell to be treated and assaying for treatment, which will vary with the pathology, or for screening for new drugs and combinations. In one aspect, the cell or tissue is obtained from the subject or patient by biopsy. This disclosure also provides kits for determining whether a pathological cell or a patient will be suitably treated by this therapy by providing at least one composition of this disclosure and instructions for use.

The test cells can be grown in small multi-well plates and is used to detect the biological activity of test compounds. For the purposes of this disclosure, the successful ELP or other agent will block the growth or kill the cancer cell but leave the control cell type unharmed.

### Combination Treatments

Administration of the therapeutic agent or substance of the present disclosure to a patient will follow general protocols for the administration of that particular secondary therapy, taking into account the toxicity, if any, of the treatment. It is expected that the treatment cycles would be repeated as necessary. It also is contemplated that various standard therapies, as well as surgical intervention, may be applied in combination with the described therapy.

As is apparent to those skilled in the art, the combination therapy can take the form of a combined therapy for concurrent or sequential administration.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the disclosure.

### EXAMPLE S

Delivery of small potent molecules to target tissue remains challenging due to uncontrolled drug distribution to healthy tissues, fast clearance and lack of biodegradable drug carrier for long circulation. To address this, Applicant provides a simple concept of genetically attaching drug's target receptor to recombinant protein polymers as its carrier. The drug interacts very strongly to its native receptor, so attaching the receptor to the protein polymer provides high affinity interaction devoid of any chemical synthesis. For example, Applicant used intact human FKBP protein domain which is the native receptor for rapamycin and second generation rapalogues. The resultant FKBP fused to protein polymer carrier after drug binding remains soluble and maintains a pharmacokinetic favorable particle size of 10 nm. This provides a rationale for using these carriers for subcutaneous administration with systemic absorption through lymphatic system. One similar formulation consists of two FKBP domains fused to both the N and C terminus of the protein polymer. This bi-headed design provides twice the drug binding and loading capacity without changing the physicochemical properties of protein polymer. Both the fusion carriers when tested in vivo in a mouse xenograft model provides long drug circulation, no peripheral tissue toxicity and cytostatic efficacy as a cancer therapy formulation. Free drug without the FKBP fusion carrier causes considerable local toxicity at site of subcutaneous injection. The concept of fusing drug receptor to protein polymers can be applied to any class of small molecules with an accessible human cognate target. This disclosure provides a new approach to deliver small molecules for subcutaneous administration on top of previously shown intravenous administration which provides more clinical relevance towards patience compliance. In a nutshell, this approach can be applied to increase tolerated dose of the drug, reduce any toxic effects associated with administration of free drug, and increase the circulation time of the drug in vivo. In addition, Applicant has observed that by engineering the attachment density and phase behavior of these fusion proteins, Applicant can extend their release over a period of months. Based on this approach, Applicant believes it may be possible to achieve long-duration delivery of potent small molecules with injections on a weekly or monthly basis, which would have significant applications in the development of new drug formulations.

Rapamycin is a highly potent and equally non-tolerable drug. With its cytostatic ability to prevent tumor growth, its application in human cancer treatment is limited by its poor bioavailability, rapid clearance and severe toxicity. With aim to synthesize a soluble long circulating rapamycin formulation, Applicant has recently reported rapamycin encapsulation by an ELP nanoparticle fused to the drug's native receptor, FKBP (FSI). The FSI-Rapa formulation completely suppressed tumor growth in vitro and in vivo and exhibited considerably reduced side effects and longer drug half-life compared to free drug. To further elucidate the implications of ELP backbone on rapamycin circulation and clearance, this article describes the influence of FKBP-ELP carriers with varying architecture and physicochemical properties on rapamycin binding, release, in vivo bio-distribution and efficacy in a xenograft mouse model.

### Characterization of FKBP-ELP Fusion Polymers

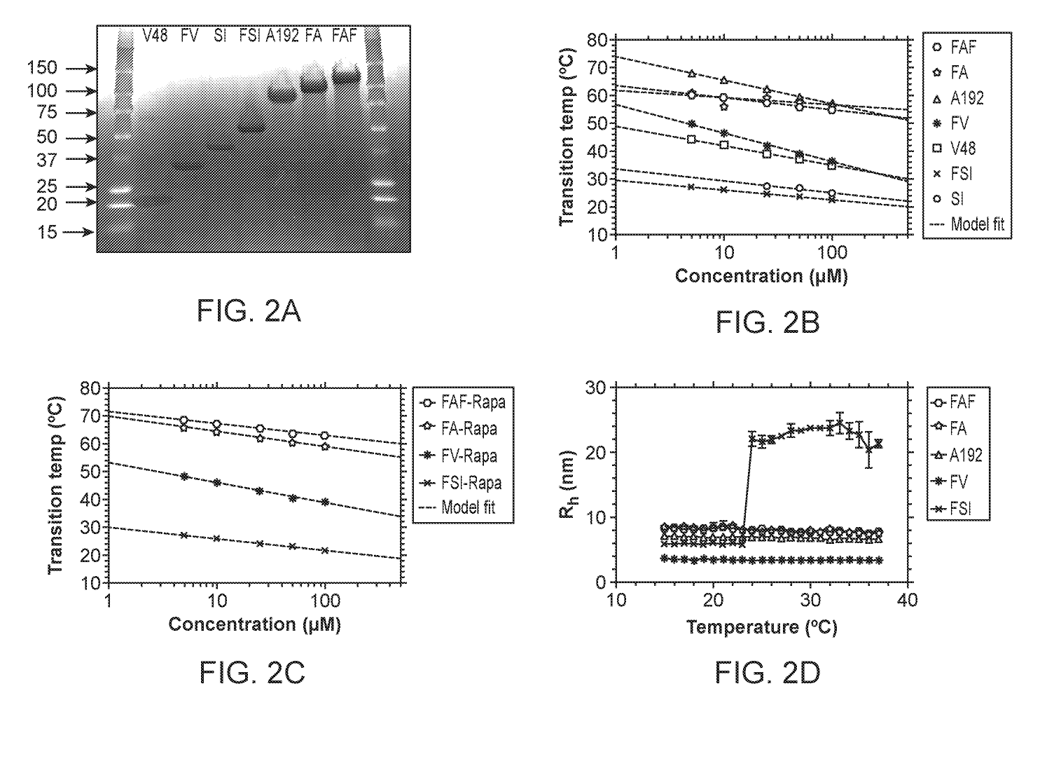

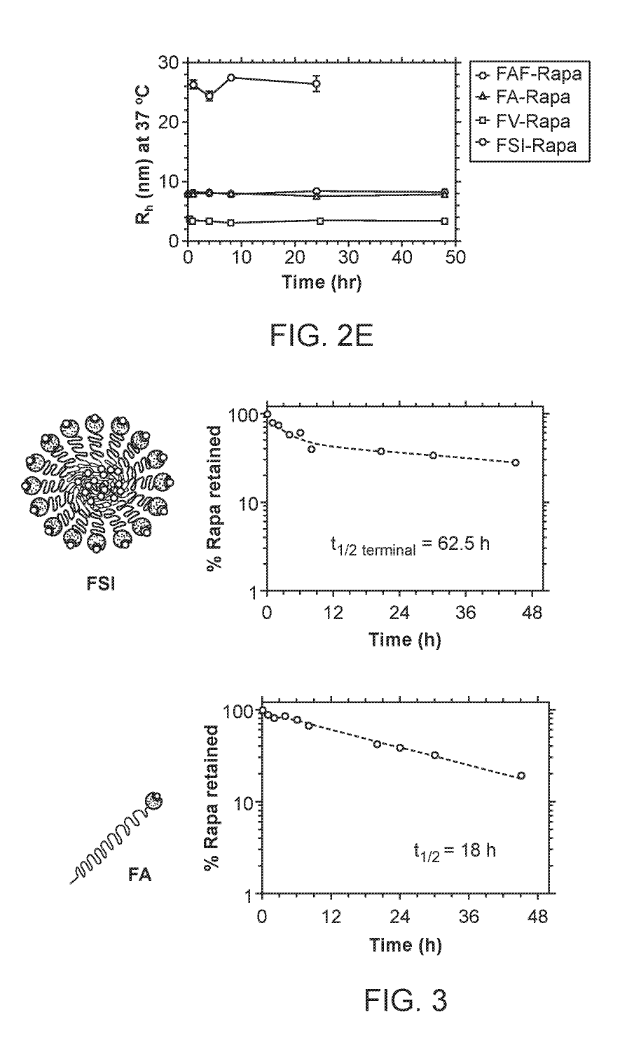

To verify the effect of ELP architecture on rapamycin bio-distribution and in vivo efficacy, second generation FKBP-ELP carriers with different molecular weight, particle size, valency and architecture were expressed and purified recombinantly from E. coli. Compared to the previously reported FKBP-S48I48 (FSI) nanoparticles, the second generation FKBP-ELP carries: FV, FA and FAF, are comparatively smaller in particle size, with molecular weight ranging from 30-100 kDa and monomeric with single guest residue composition, in other words, devoid of forming nanoparticles (Table 1). SDS-PAGE gel was used to determine the purity of all the FKBP-ELPs along with their respective ELP backbones (FIG. 2A). Optical density measurement was used to study ELP phase separation property with and without FKBP (FIG. 2B). As observed in the figure, fusing FKBP to ELP minimally influenced the ELP transition temperature and Tₜ follows an inverse relationship with logarithm of the ELP concentration. Similar ELP phase transition profile was evaluated with all FKBP-ELPs after rapamycin encapsulation (FIG. 2C) which elucidates no drug interference on the thermal properties of FKBP-ELPs. Using the fit equation parameters for FKBP-ELPs (Table 1, FIG. 2B), it is possible to estimate solubility profiles of all FKBP-ELPs at 37° C. across a concentration range of 1-500 μM. The data points were fit to linear equation Tₜ=b−m [Log₁₀(concentration)], to obtain a non-linear regression model fit indicated as the dotted line. At 37° C., FA and FAF remains completely soluble, FSI assembles into distinct nanoparticles, while FV remains soluble at concentrations ≤200 μM.

After studying the thermal properties of FKBP-ELPs, the hydrodynamic radius (Rₕ) of all the constructs was evaluated using Dynamic Light Scattering (FIG. 2D). As shown in the figure, (i) particles were minimally influenced by fusing FKBP to respective ELP backbones and (ii) monomeric FV, FA and FAF remain soluble at physiological temperatures whereas FSI diblock copolymer assembles into nanoparticles at 25° C. Further, stability of FKBP-ELPs was evaluated by measuring the particle size after rapamycin encapsulation for a period of 48 h at 37° C. (FIG. 2E). The particles remained stable with same particle size which shows that drug binding to FKBP doesn't alter the particle size and had no effect on particle morphology.

### Bivalent Soluble FAF Prolongs Rapamycin Release by 30 Fold Over FSI Nanoparticles

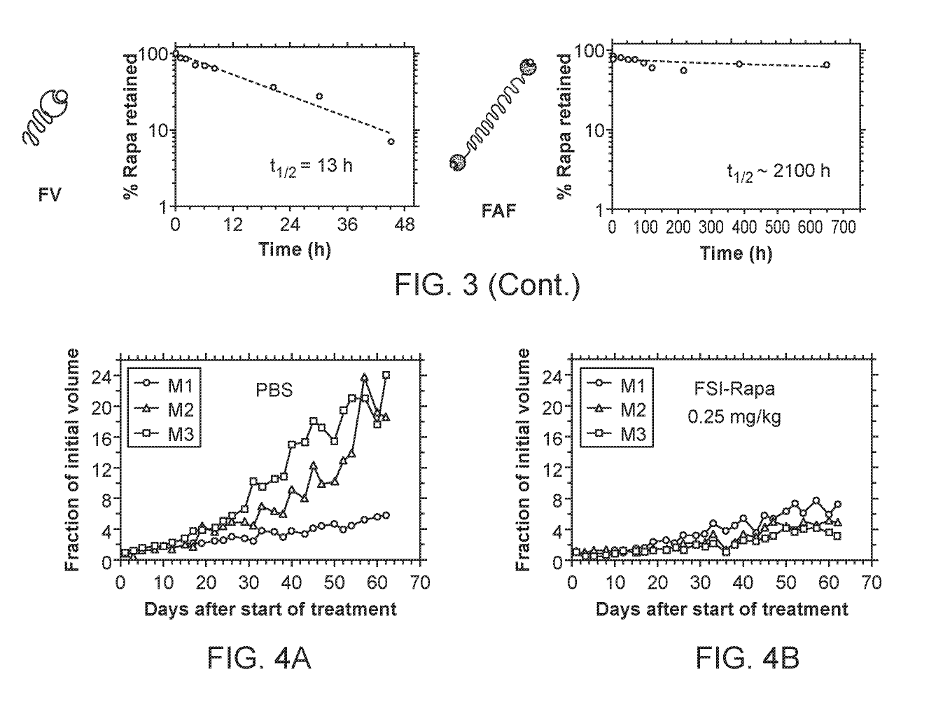

After performing the physicochemical evaluation, all the FKBP-ELPs were tested for rapamycin release post drug encapsulation (FIG. 3). FV, FA and FSI showed encapsulation efficiency ˜70-90% whereas FAF showed ˜160% drug binding because of the FKBP bivalency per ELP. The dialysis was performed under sink conditions in PBS containing 1× pencillin-streptomycin at 37° C. for all the FKBP-ELPs except 30° C. for FV to prevent ELP phase separation. FSI showed a two phase exponential release with a shorter half-life of 2 hours and a longer terminal half-life of 60 hours as reported previously. On the other hand, monomeric soluble FKBP-ELPs, FV and FA showed a single fast release half-life of ˜13 hours. This study showed consistent findings with our previous report of nanoparticle assembly giving a longer rapamycin half-life compared to monomeric soluble FV. A possible explanation behind the significantly different release rates could be the dense brush of FKBP at the corona of FSI nanoparticles which provides a sustained drug release over time. Whereas FV and FA do not assemble into nanoparticles and without any ELP based assembly design, the release rate from FKBP domain remains fast. This further elucidates that ELP hydrophobicity, length and molecular weight has no impact on the drug release profile and that the release of drug from soluble monomeric FKBP-ELPs remains constant. In contrast, when the experiment was repeated with bivalent FAF, an apparent terminal prolonged half-life of ˜3 months was observed with 80% drug retention after a month of dialysis. The mechanism behind the delayed release may reflect the co-operativity between terminal FKBPs of FAF molecules producing a hydrophobic binding pocket for rapamycin which remains stable in the absence of any protease activity. This observation further shows the temperature independence and stability of FAF to retain drug at 37° C. for extended period of time which could make possible ease of storage with longer shelf-life. All the FKBP-ELPs showed high drug encapsulation efficiencies but displayed a varying drug release profile depending upon the ELP architecture, solubility and FKBP valency. From this comparative study, FSI was found to have a 3 fold longer terminal drug half-life compared to FV and FA whereas FAF showed a 30 fold longer half-life compared to FSI nanoparticles which may prevent release of free drug and provide comparatively longer circulation in vivo.

FSI Nanoparticle Assembly is not Required for Suppressing Tumor Growth In Vivo when Administered Intravenously

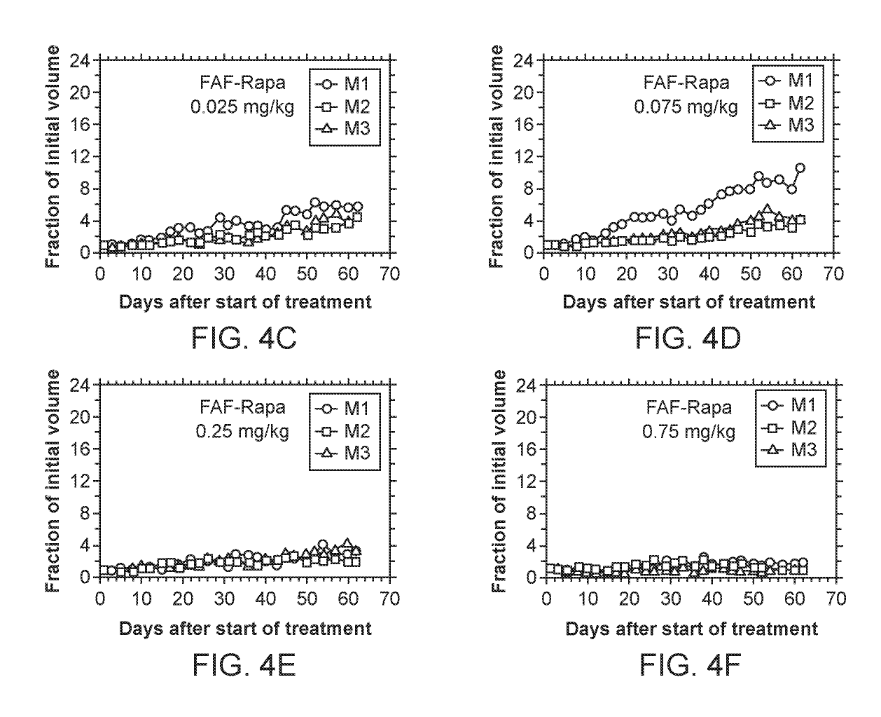

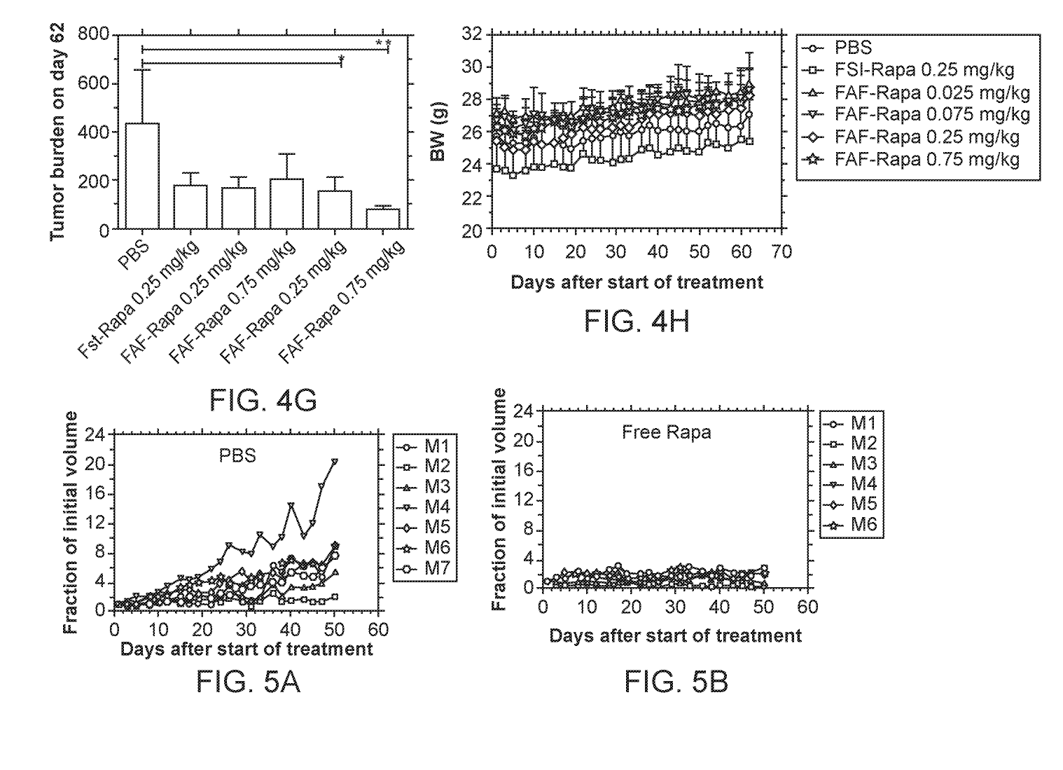

After characterizing the drug release from all the FKBP-ELPs, these carriers were evaluated for in vivo efficacy by IV administration in a MDA-MB-468 breast cancer xenograft mouse model (FIGS. 4A-4H). A tumor regression study consisting of 6 groups was performed with a dose escalation regimen of FAF-Rapa at 0.025 mg/kg, 0.075 mg/kg, 0.25 mg/kg and 0.75 mg/kg along with PBS and FSI-Rapa at an intermediate dose of 0.25 mg/kg (n=3/group). FAF was chosen for in vivo dose escalation compared to other FKBP-ELPs for its favorable systemic physicochemical properties including high molecular weight, small particle size and prolonged drug release profile. Applicants have previously reported complete tumor suppression in the same xenograft mouse model with FSI-Rapa at maximum tolerated dose of 0.75 mg/kg when given I.V. The rationale for performing this dose escalation tumor study was to test the effect of soluble FAF architecture verses FSI nanoparticle assembly on drug's in vivo efficacy at a fixed dose and simultaneously get a future perspective on efficacy of FAF at low-high doses of rapamycin. All treatments were done intravenously by tail vein injections.

All the formulations were well tolerated by the mice with no decrease in body weight. However, with respect to efficacy in comparison to PBS group, only the groups treated with FAF-Rapa at 0.25 mg/kg and 0.75 mg/kg responded statistically (FIG. 4G). The treatment groups analyzed by performing 1-way ANOVA on the mean±SD tumor burden on the last day of treatment showed significant difference between all the 6 groups (α=0.05 with 95% CI, P=0.0166). Tukey's multiple comparison post hoc analysis was performed to test significance between the individual groups. Groups treated with FAF-Rapa at low doses of 0.025 mg/kg and 0.075 mg/kg showed partial response with tumor volume reaching 4-6 times its initial volume by end of treatment and showed no statistical significance compared to PBS (α=0.05 with 95% CI). A similar response was observed with FSI-Rapa at 0.25 mg/kg with no statistically significant difference compared to PBS group (α=0.05 with 95% CI, P=>0.05). However, the groups treated with FAF-Rapa at 0.25 mg/kg and 0.75 mg/kg responded to the treatment with tumor volume being less than 4 times its initial volume and statistically significant difference was observed compared to PBS group (α=0.05 with 95% CI).

Even though tukey's post hoc analysis revealed significant difference between PBS and FAF-Rapa at 0.25 mg/kg, there was no significant difference between FAF-Rapa and FSI-Rapa at 0.25 mg/kg (α=0.05 with 95% CI,). This suggests that even though FAF and FSI differ in their assembly and physicochemical properties with different drug release profiles, there was no significant difference in rapamycin efficacy in vivo with the respective carriers. This may suggest a different release mechanism of the drug from systemic FKBP-ELPs which alters the drug's pharmacokinetics. A plausible explanation is plasma protein albumin binding to rapamycin and dissociating the drug off the FKBP binding pocket, nullifying the effect of ELP assembly and architecture on drug's efficacy in vivo. The similar tumor burden seen with FAF-Rapa and FSI-Rapa at 0.25 mg/kg suggests the likelihood of a similar mechanism. However, the effect of albumin on drug release from FKBP-ELPs remains to be addressed in future studies. Based on the dose escalation data, FAF-Rapa can suppress tumor growth in vivo similar to FSI-Rapa reported previously. But given the positive features of temperature independency, enhanced stability, and half the stoichiometric ELP required for drug loading due to the bi-valency effect, FAF remains a better candidate than FSI for IV administration.

FAF Outperforms Other FKBP-ELPs in Suppressing Tumor Growth In Vivo when Administered Subcutaneously

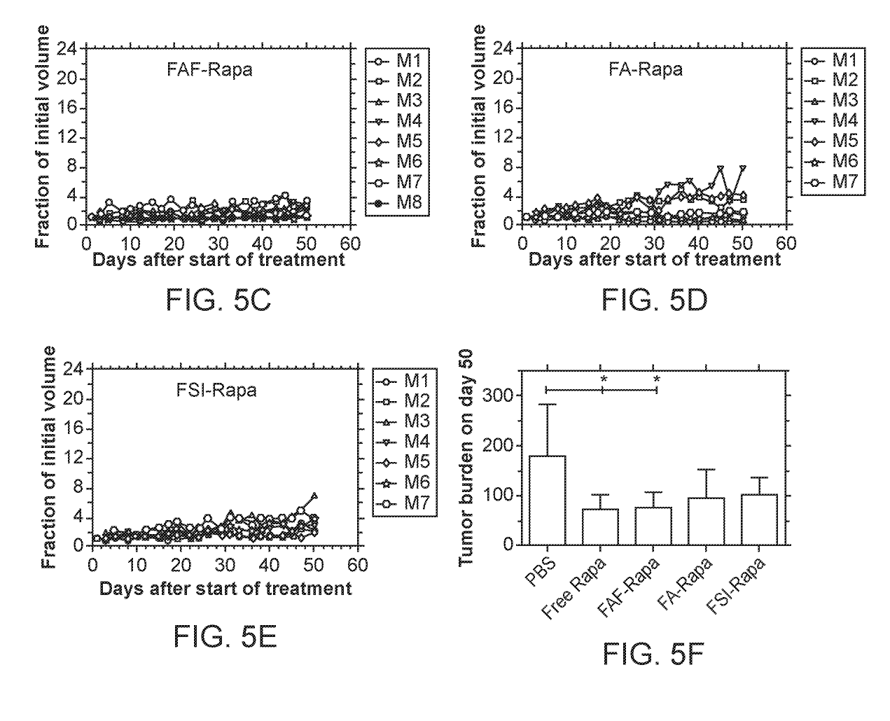

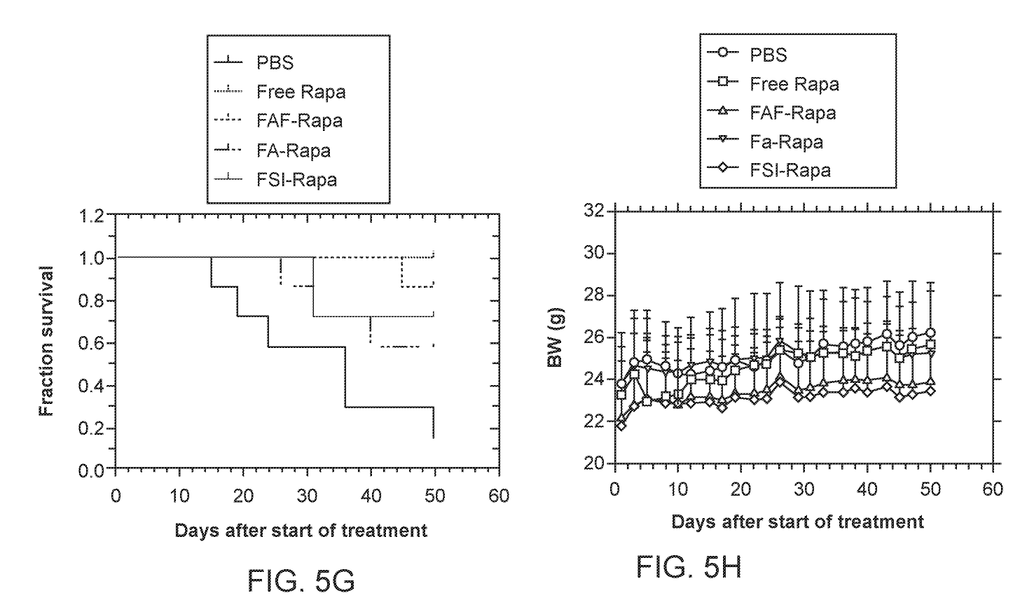

Since there was no statistical difference in efficacy observed between FSI-Rapa and FAF-Rapa intravenously, Applicants next employed SC route of administration in the same MDA-MB-468 xenograft mouse model (FIGS. 5A-5H). The rationale behind exploring SC route was to distinguish the uptake and bio distribution of different FKBP-ELPs from the site of injection into the systemic circulation. Bioavailability through the SC route is governed by transport through the interstitium into the lymphatic system which has been shown to depend on factors like molecular weight, particle size and surface charge of biotherapeutics. This provided a platform to study bio-distribution of drug loaded FKBP-ELPs via the SC route which displays a wide range in molecular weight, particle size and assembly properties (Table 1). SC route further presents a convenient administration method compared to IV route with more clinical relevance and has been FDA approved for monoclonal antibodies and biotherapeutics. Glymera™, a subcutaneous ELP formulation is currently under PhaseIIB status as a therapeutic for Type II diabetes which shows evidence of ELP absorption into systemic circulation from site of injection.

The comparative tumor regression study consisted of 5 groups: PBS, Free Rapa, FAF-Rapa, FA-Rapa, and FSI-Rapa at the same maximum tolerated dose of 0.75 mg/kg (n=6-8/group). All treatments were done subcutaneously in the right flank above the hind leg. Free Rapa was administered in 100% DMSO for the first week of treatment which produced bleeding and bruising at the site of injection and a 10% drop in BW. Because of the toxic side effects, mice were thereafter treated with free Rapa in DMSO:EtOH:Cremophore-EL:PBS formulation in 1:1:2:6% v/v ratio. Even after reducing the organic solvent content to 20%, local bruising and scabs were observed at the site of injection which remained fresh for a week's interval. However, the mice gradually gained back the lost BW which enabled us to continue the treatment with the Cremophore formulation. To prevent further local discomfort to mice, a different site of injection was chosen each time spanning the entire flank region for free Rapa injections. In contrast to free Rapa, all the other FKBP-ELPs showed no signs of toxicity throughout the duration of the treatment. There was no local bruising or scabs observed with any of the FKBP-ELP-Rapa formulations which made the treatment easier and at the same site of injection throughout the study.

Compared to PBS group, only Free Rapa and FAF-Rapa responded statistically to the treatment (FIGS. 5A-5H). Similar to the IV study, the treatment groups were analyzed by performing 1-way ANOVA on the mean±SD tumor burden on the last day of treatment and statistical significance was observed between all the 5 groups (α=0.05 with 95% CI, P=0.0113). Tukey's multiple comparison post hoc analysis revealed FSI-Rapa and FA-Rapa having no statistical significance compared to PBS (α=0.05 with 95% CI). However, the groups treated with Free Rapa and FAF-Rapa demonstrated statistically significance compared to PBS group (α=0.05 with 95% CI). Kaplan-Meier analysis was employed to distinguish the survival rates with 4 times increase in tumor volume as the endpoint (FIG. 5G). Log Rank (Mantel-Cox) test performed on the survival analysis revealed significant difference between the survival curves (P=0.0021). Based on these findings, only Free Rapa and FAF-Rapa showed significance in vivo efficacy compared to PBS. However, FAF-Rapa had no any local toxicity at the site of injection.

FA and FAF Demonstrates Better Bio Distribution and Tumor Accumulation than FSI Nanoparticles.

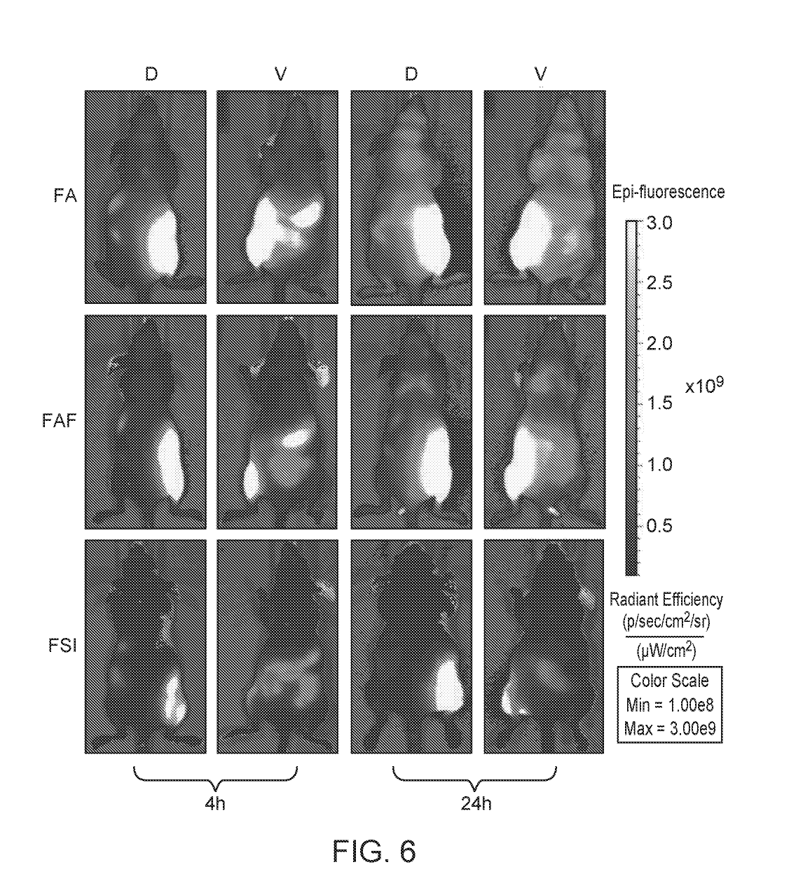

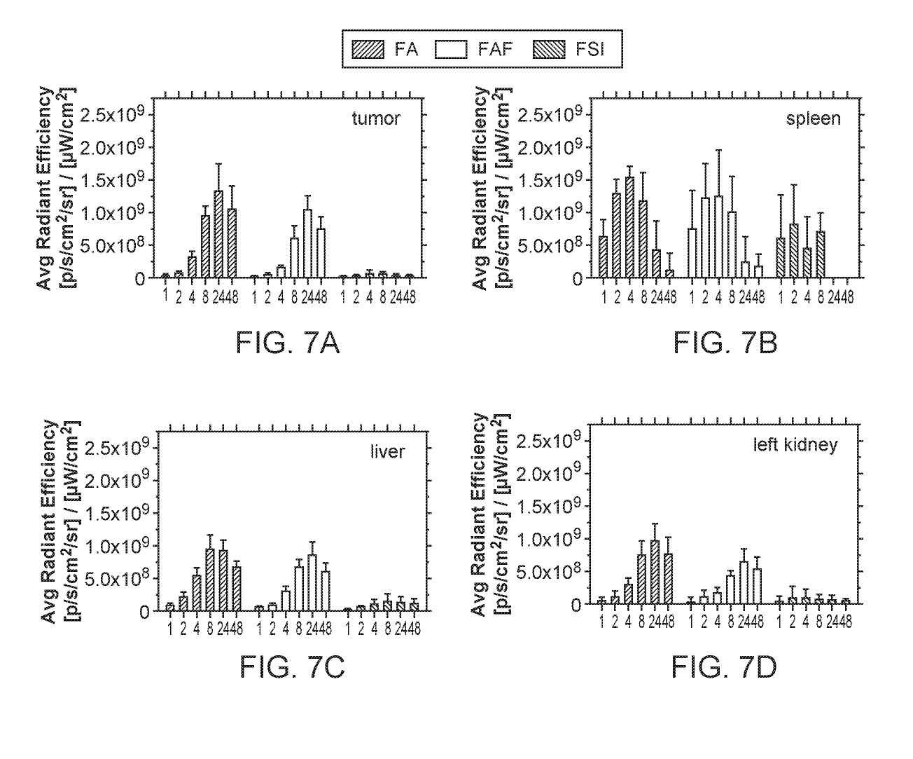

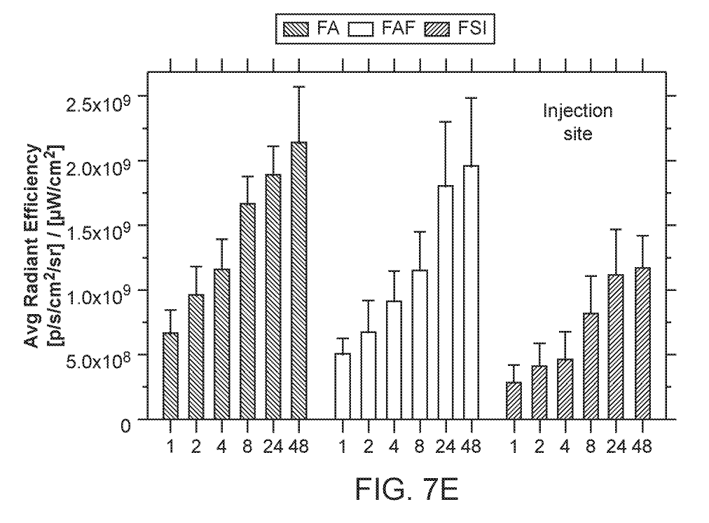

To further understand the effect of ELP architecture and assembly on the SC absorption of FKBP-ELPs, Applicants next studied its bio-distribution in xenograft nude mice using a near infrared dye, Cyanine5.5 (FIG. 6). FA, FAF and FSI were labelled with Cyanine5.5 using NHS chemistry and administered subcutaneously in the flank above the right leg (n=4/group). Bioluminescence was monitored for a period of 48 hours by taking dorsal and ventral scans using IVIS spectrum (Perkin Elmer) at predetermined fixed scan intervals (FIGS. 7A-7E). As observed in the figure, FA and FAF showed gradual increase in tumor accumulation which peaked at 24 hours. The gradual increase in tumor signal for FA and FAF was found to correlate with high initial spleen signal which peaked at 8 hours followed by rapid decline thereafter. The liver and kidney signal for FA and FAF were also found to peak at 24 hours in correlation with low spleen signal at similar intervals suggesting liver and kidneys to be the clearance organs for FA and FAF. FSI signal was found to be very weak in the tumor and similar to FA and FAF, FSI showed high initial spleen accumulation followed by rapid decline at later time points. However, liver and kidney accumulation for FSI was very low. Bioluminescence at the injection site was found to increase with respect to time with more diffusion seen with FA and FAF than FSI which could be due to accumulation of FKBP-ELPs in the surrounding interstitial lymph nodes.

Absorption of biologics from SC site into systemic circulation has been well characterized in literature to occur either by direct uptake by blood capillaries or by lymphatic vessels. Biologics with particle size <10 nm and MW≤16 kDa are more easily taken up by blood capillaries. Whereas high MW protein therapeutics with particle size in range of 10-100 nm are taken up by lymphatic vessels. FA, FAF and FSI display high molecular weight in range of 50-100 kDa and particle size in range of 9-25 nm (Table 1, FIG. 2) which both are characteristics of uptake by lymphatic vessels.

### FKBP-ELP and FKBP-ELP-FKBP Gene Design and Cloning.

FKBP-ELP cloning was done as previously described. Cloning of FKBP-ELP-FKBP was done by attaching FKBP gene onto C terminus of FKBP-ELP. FKBP gene that goes on the C terminus was ordered in an ampicillin resistant proprietary pIDTsmart vector (Integrated DNA technologies) with three restriction cut sites: XbaI, BserI and BamHI. The vector was designed such that FKBP gene was flanked with cut sites for BserI and BamHI with XbaI and BamHI at the 5′ and 3′ prime ends of the FKBP gene respectively. The IDT vector was double digested with XbaI and BamHI and the FKBP gene was isolated by running a 1% agarose gel electrophoresis. The FKBP gene was then inserted into empty pET25b (+) vector (Novagen) digested with same set of enzymes. With a second cloning step, the pET25b (+) vector having the FKBP gene was then digested with BserI and BssHII and was ligated to C terminus of FKBP-ELP gene (cloned previously) isolated from pET25b (+) vector by digesting with AcuI and BssHII. The in-frame amino acid sequence was confirmed by DNA sequencing.

### FKBP-ELP Expression and Purification.

The pET25b(+) vectors with FKBP-ELP or FKBP-ELP-FKBP gene were transfected into BLR (DE3) E. coli competent cells (Novagen) and plated onto Agar plates with 100 μg/mL ampicillin and incubated overnight in a 37° C. incubator. 5-6 colonies were picked for each construct and evaluated for highest protein expression by transforming each colony into 50 mL Terrific Broth (TB) media grown overnight supplemented with 100 μg/mL carbenicillin at 37° C. The bacterial culture grown from each colony was amplified to 1 L TB media supplemented with 100 μg/mL carbenicillin and allowed to grow for 24 hours at 37° C. The culture was centrifuged and pellet was resuspended in Dulbecco's sterile phosphate buffered saline (PBS) buffer (Corning). The resuspension was subjected to tip probe sonication for cell lysis. The supernatant containing fusion protein was purified using Inverse Transition Cycling (ITC) as previously described. The colony having the highest protein expression was purified in bulk in 8-9 L TB media with yield of 50-60 mgs/L. The purified protein was filtered through 200 nm sterile acrodisc 25 mm filters (Pall Corporation) and estimated for its concentration using Beer Lambert's law:

$$Protein concentration ( M ) = \frac{( {A}_{280} - {A}_{350} ) \times dilution factor}{MEC \times l}$$

where A₂₈₀ and A₃₅₀ are absorbance at 280 and 350 nm respectively, l is the path length (cm) and MEC is the estimated molar extinction coefficient at 280 nm, 11585 M⁻¹cm⁻¹ for FKBP-ELP and 20190 M⁻¹cm⁻¹ for FKBP-ELP-FKBP.

### FKBP-ELP Physicochemical Characterization

The purified fusion proteins were characterized for its physicochemical properties by measuring purity using SDS-PAGE gel, optical density using UV-Vis spectrophotometer and particle size using Dynamic Light Scattering. Purity of ELPs was determined by running denatured samples on 4-20% gradient Tris-Glycine-SDS PAGE gel. 6-12 μg protein in water was mixed with SDS loading buffer containing 10% β-mercapto ethanol and heated at 90° C. for 5 mins before loading onto the gel. Gels were stained using 10% w/v copper chloride solution and imaged using BioRad Gel Imager (FIG. 2A).

The temperature-concentration phase diagrams were obtained by measuring optical density profiles at 350 nm as a function of temperature on a UV-Vis DU 800 spectrophotometer. A temperature ramp was performed by heating different concentrations of ELPs (with and without FKBP) and FKBP-ELPs (with and without rapamycin) in Beckman Coulter Tm microcells (Brea, Calif.). The temperature was increased at rate of 1° C./min with readings taken every 0.3° C. increment. The increase in optical density was analyzed by first derivative and the corresponding temperature showing the highest first derivative was defined as the phase transition temperature (FIGS. 2B-2C).

The particle size of all the FKBP-ELPs was evaluated by measuring Hydrodynamic radius (Rₕ) using Dynamic Light Scattering. 25 μM concentration samples were filtered through 200 nm sterile acrodisc 13 mm filters (Pall Corporation) and 60 μL of each sample was loaded in triplicates in 384 well plate (Greiner Bio One) and covered with 20 μL mineral oil. The plate was centrifuged at 3000 rpm for 3 minutes to clear any surface air bubbles. All samples, tips, and eppendorff tubes were kept chilled at 4° C. before analysis. Samples were analyzed using Wyatt Dynapro plate reader (Santa Barbara, Calif.) from 20-37° C. at interval of 1° C. (FIG. 2D). The stability of FKBP-ELPs was evaluated at 37° C. for period of 48 hours after rapamycin encapsulation to study the effect of rapamycin binding on the size and structural properties of FKBP-ELPs (FIG. 2E). The reported values are presented as mean±SD.

### Rapamycin Encapsulation and Formulation for In Vivo Injections.

Purified and characterized FKBP-ELPs were used for rapamycin encapsulation using two-phase solvent evaporation method. 200-400 μM (2 mL) FKBP-ELP in PBS was equilibrated in a glass vial at room temperature (37° C. for FSI nanoparticles) for 5-10 minutes followed by addition of excess rapamycin in hexane/EtOH mixture (70/30%) to obtain a two phase mixture of FKBP-ELP in PBS at bottom and rapamycin in hexane at the top. The organic phase was evaporated under mild flow of N₂ gas with continuous stirring on a magnetic stir plate for 20 minutes. After complete evaporation of organic solvent, the remaining aqueous solution was centrifuged at 13000 rpm at room temperature (37° C. for FSI nanoparticles) to obtain pellet of free rapamycin. The supernatant was filtered through 200 nm sterile acrodisc 25 mm filters (Pall Corporation) and injected into a C-18 RP-HPLC column (Waters, Inc.) for rapamycin quantification bound to FKBP-ELP. FSI-Rapa solution was dialyzed in PBS for 12 hours to remove rapamycin bound non-specifically to the hydrophobic core of FSI nanoparticles. Post quantification, all drug loaded FKBP-ELP formulations were diluted to required mg/kg mouse body weight dose and aliquots for single use were stored frozen at −80° C.

Rapamycin Release from FKBP-ELPs by Dialysis

Rapamycin encapsulation to FKBP-ELPs and quantification was done as explained previously. Drug loaded FKBP-ELPs were dialyzed in PBS at room temperature (37° C. for FSI-Rapa nanoparticles) with continuous stirring to monitor the release rate. 2 mL solution was dialyzed in 20 kDa MW cutoff cassettes (Thermo Scientific) under 1:750 sink conditions. Penicillin-Streptomycin 1× solution was added to PBS to prevent bacterial contamination. 100 μL aliquots were collected from the cassette at fixed time intervals and each aliquot was quantified for rapamycin concentration using RP-HPLC. The rapamycin concentrations at different time points were normalized to % drug retained compared to 0 h drug concentration and release half-life was calculated by performing non-linear regression. The data is presented as mean±SD with n=3 assays for each FKBP-ELP (FIG. 3).

### In Vivo Tumor Regression Study.

All animal experiments were conducted as per the guidelines of the American Association of Laboratory Animal Care under an USC approved protocol. MDA-MB-468 cells (American Type Tissue Culture Collection) were cultured at 37° C. with 5% CO₂ in Dulbecco's modified Eagle's medium (DMEM)/Ham's F-12 media (Caisson) supplemented with 10% fetal bovine serum. The cells were sent for screening against all major mouse pathogens and human blood borne pathogens (Charles River) prior to implantation. A single injection of MDA-MB-468 cells (1-2×10⁶ cells in 100 μL fetal bovine serum free DMEM media) was implanted into the left mammary fat pad of 7-8 weeks old female nude (nu/nu) athymic mice (Harlan, Inc.) Tumors were allowed to grow to a size of ˜50-100 mm³ and mice were randomized blindly into respective groups before start of treatment. Mice were injected intravenously via tail vein or subcutaneously in the flank above the hind leg with free rapamycin or rapamycin loaded FKBP-ELPs thrice a week on Mondays, Wednesdays and Fridays. BW and tumor size were measured thrice a week on the same day of injections. Tumor size was measured using Vernier calipers and tumor volume was calculated using the following equation:

$$Tumor volume = \frac{{a}^{2} \times b \times π}{6}$$

where a and b represents smaller and bigger length respectively. Two independent tumor regression studies were performed. First study comprised of a FAF-Rapa dose escalation study of 6 groups with n=3 in each group (FIGS. 4A-4H). The 6 groups were treated intravenously with 100 μL injections of PBS, FAF-Rapa (0.025 mg/kg), FAF-Rapa (0.075 mg/kg), FAF-Rapa (0.25 mg/kg), FAF-Rapa (0.75 mg/kg) and FSI-Rapa (0.25 mg/kg). The second tumor regression study comprised of a comparative study of 5 groups where free Rapa, FAF and FA and FSI were compared at a fixed dose of 0.75 mg/kg with n=6-8/group (FIGS. 5A-5H). The 5 groups were treated subcutaneously with 150-200 μL injections of PBS, Free Rapa (100% DMSO or DMSO:EtOH:Cremophore-EL:PBS in 1:1:2:6% v/v ratio, 0.75 mg/kg), FAF-Rapa (0.75 mg/kg), FA-Rapa (0.75 mg/kg), and FSI-Rapa (0.75 mg/kg). Mice were euthanized at humane end point of 1000 mm³ tumor volume. Tumor regression data from both the studies is presented as fraction of initial tumor volume for each mouse in all the groups and as tumor burden with mean±SD for each group. Tumor burden was calculated by determining the trapezoidal area under the growth curve for each mouse by using the following equation:

$$Trapezoidal area = \sum \frac{[ ( {T}_{f 1} + {T}_{f 2} ) \times h ]}{2}$$

where T_{f1} is fraction of initial tumor volume on day 1, T_{f2} is the fraction of initial tumor volume on day 3, and h is the difference between consecutive days. For Kaplan-Meier survival analysis, tumor reaching four times its original volume was considered as the end point. Body weights are shown as mean±SD.

### Histopathological Examination of Rapamycin Treated Mice

At the end of subcutaneous tumor regression study, mice from all the groups were euthanized and organs were collected for histopathological examination. Before performing euthanasia, mice were anaesthetized with 2% v/v isoflurane gas with oxygen for whole body blood perfusion. Heart, lungs, spleen, liver, kidney and skin were excised, and fixed in zinc formalin overnight before preserving into 70% ethanol. Appropriate size cut organs were then processed for paraffin embedding followed by mounting 5 μm tissue slices on glass slides and staining with hematoxylin and eosin (H & E). The H & E stained tissues were then studied under microscope for histopathological changes.

### Bioluminescence Imaging of Fluorescently Labelled FKBP-ELPs

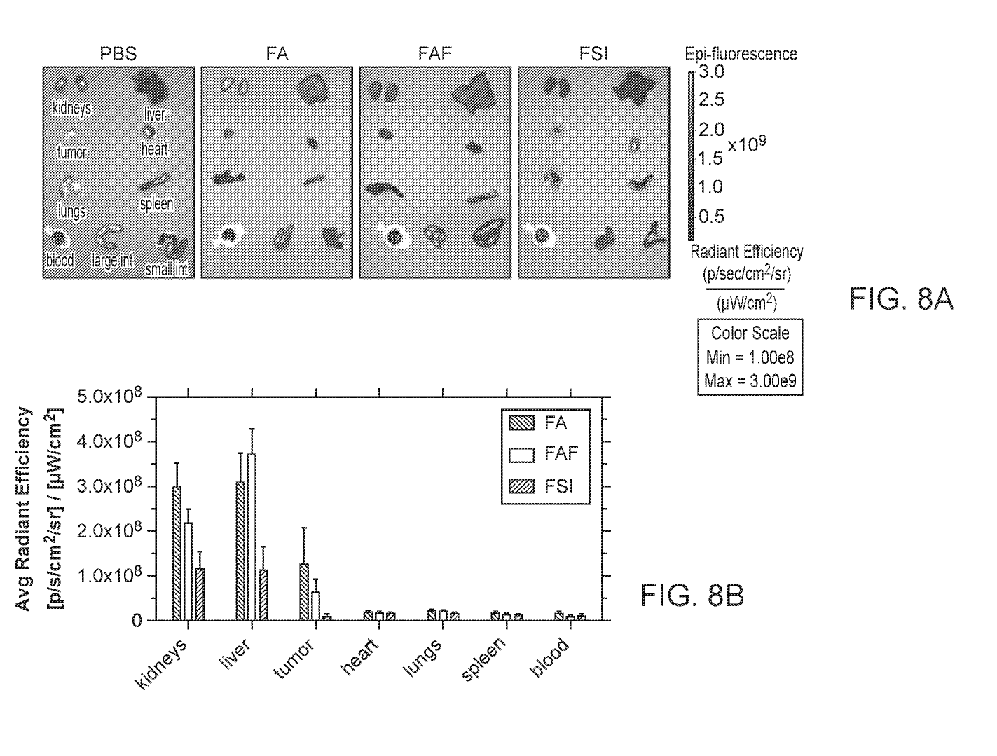

All animal experiments were conducted as per the guidelines of the American Association of Laboratory Animal Care under an USC approved protocol. Tumor implantation was performed as described in the tumor regression study. FAF, FA and FSI were labelled with near infrared Cyanine5.5 dye using NETS-chemistry and were administered subcutaneously in the right flank above the hind leg (n=4/group). Mice were anaesthetized with 2% v/v isoflurane gas with oxygen followed by SC injection of Cyanine5.5 labelled FKBP-ELPs. Whole body dorsal and ventral scans were imaged using the IVIS optical spectrum (Perkin Elmer) at 0,1,2,4,8,24 and 48 hour post injection using 1 second exposure time and small binning (FIG. 6). Excitation and emission filter for Cyanine5.5 were chosen to be 640 nm and 700 nm respectively. Images were analyzed using Living Image® (Perkin Elmer) software. Region of Interest (ROI) was drawn on tumor, liver and spleen from ventral scans and that of left kidney and injection site from dorsal scans. Fluorescence from respective ROIs was quantified in average radiance efficiency with units (photons/sec/cm²/sr)/(μW/cm²) and plotted after subtracting respective ROI background intensity at 0 hr. Organ distribution of Cyanine5.5 labelled FKBP-ELPs with respect to time is presented as mean±SD with n=4 per group (FIGS. 7A-7E). After 48 hours, mice were euthanized and small volume of blood was withdrawn via cardiac puncture. Mice were then skinned and imaged dorsally and ventrally for any evidence of lymph node accumulation. The carcasses were then dissected and individual organs along with blood withdrawn earlier were scanned using 1 second exposure time with small binning. The fluorescence from the dissected organs was quantified as described earlier (FIGS. 8A-8B).

TABLE 1
Physicochemical properties of ELP protein polymers with and without FKBP

| | | | Rₕ at | Rₕ at | ^{b}Slope, | |
|---|---|---|---|---|---|---|
| | | Expected | 20 C. ° | 37 C. ° | in [° C. | ^{b}Intercept, |
| Label | ^{a}Amino acid sequence | MW (kDa) | (nm) | (nm) | Log (μM)] | b (° C.) |
| V48 | MG(VPGVG)₄₈Y | 19.7 | | | 7.01 | 48.8 |
| FKBP-V48 (FV) | FKBP-G(VPGVG)₄₈Y | 31.5 | 3.6 | 3.6 | 10.26 | 56.7 |
| S48I48 (SI) | MG(VPGSG)₄₈(VPGIG)₄₈Y | 39.6 | 4 | 24 | 4.1 | 33.3 |
| FKBP-S48I48 (FSI) | FKBP-G(VPGSG)₄₈(VPGIG)₄₈Y | 51.4 | 5 | 25 | 3.5 | 29.4 |
| A192 | MG(VPGAG)₁₉₂Y | 73.5 | 7 | 7 | 8.4 | 73.9 |
| FKBP-A192 (FA) | FKBP-G(VPGAG)₁₉₂Y | 85 | 8 | 8 | 2.5 | 61.6 |

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosure embodied therein herein disclosed can be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. An agent comprising an elastin-like peptide (ELP) component, a therapeutic agent and a N-terminus ligand and a C-terminus ligand conjugated to the N-terminus and C-terminus of the ELP, each ligand selected to target a receptor of the therapeutic agent, the N-terminus and the C-terminus ligand being the same or different from each other.

2. The agent of claim 1, wherein the molecular weight of the ELP-ligand is between 20 to 150 kDa.

3. The agent of claim 1, further comprising a linker between either or both of the N-terminus ligand and the C-terminus ligand and the ELP.

4. (canceled)

5. (canceled)

6. The agent of claim 1, wherein the therapeutic agent is rapamycin and the N-terminus and/or the C-terminus ligand comprises an FK506 binding protein (FKBP), or a biological equivalent thereof, wherein a biological equivalent of the FKBP protein is a peptide that has at least 80% sequence identity to FKBP or a peptide encoded by a polynucleotide that hybridizes under conditions of high stringency to a polynucleotide that encodes FKBP or its complement, wherein conditions of high stringency comprise hybridization reaction at about 60° C. in about 1×SSC and binds rapamycin.

7. The agent of claim 1, wherein the therapeutic agent is cyclosporin A and the N-terminus and/or the C-terminus ligand comprises cyclophilin A, or a biological equivalent thereof, wherein biological equivalent of cyclophilin A is a peptide that has at least 80% sequence identity to cyclophilin A or a peptide encoded by a polynucleotide that hybridizes under conditions of high stringency to a polynucleotide that encodes cyclophilin A or its complement, wherein conditions of high stringency comprise hybridization reaction at about 60° C. in about 1×SSC and binds cyclosporin A.

8. (canceled)

9. The agent of claim 1, wherein the ELP comprises reference polypeptide (VPGXG)n, (wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about 192, and X is an amino acid selected from Ser, Ala, Ile, or Val, or a biological equivalent thereof, wherein a biological equivalent of the reference polypeptide is a peptide that has at least 80% sequence identity to the reference polypeptide or a peptide encoded by a polynucleotide that hybridizes under conditions of high stringency to a polynucleotide that encodes the reference polypeptide or its complement, wherein conditions of high stringency comprise hybridization reaction at about 60° C. in about 1×SSC.

10. (canceled)

11. (canceled)

12. A method for delivering a therapeutic agent in vitro comprising contacting a tissue with the agent of claim 1.

13. A method for delivering a drug in vivo comprising administering an effective amount of the agent of claim 1 to a subject.

14. A method for ameliorating the symptoms of a disease or condition or for treating a disease or condition, comprising administering an effective amount of the agent of claim 1 to a subject suffering from the disease or condition or susceptible to the disease or condition.

15. The method of claim 14, wherein the disease or condition is selected from the group of age-related macular degeneration, Sjögren's syndrome, autoimmune exocrinopathy, diabetic retinopathy, graft versus host disease (exocrinopathy associated with) retinal venous occlusions, retinal arterial occlusion, macular edema, postoperative inflammation, uveitis retinitis, proliferative vitreoretinopathy, glaucoma, keratoconjunctivitis sicca (dry eye), scleritis or glaucomacancer, and the agent is selected to treat the disease or condition.

16. A kit for ameliorating the symptoms of a disease or condition or treating a disease, comprising an agent of claim 1, and instructions for use.

17. An isolated polynucleotide encoding an elastin-like peptide (ELP) component that forms a stable nanoparticle above the transition temperature of the ELP and an N-terminus ligand and a C-terminus ligand, wherein each ligand is selected to target a receptor of a therapeutic agent, wherein the N-terminus and the C-terminus ligand may be the same or different from each other.

18. The isolated polynucleotide of claim 17, wherein the ELP comprises a reference polypeptide (VPGXG)n, (wherein n is an integer that denotes the number of repeats, and can be from about between 5 and 400, alternatively between 5 and 300, or alternatively between 25 and 250, or alternatively between 25 and 150, or from about 6 to about 200, or alternatively from about 15 to 195, or alternatively from 40 to about 195, or alternatively about 24, or alternatively about 48, or alternatively about 96, or alternatively about 192, and X is an amino acid selected from Ser, Ala, Ile, or Val, or a biological equivalent thereof, wherein a biological equivalent of the reference polypeptide is a peptide that has at least 80% sequence identity to the reference polypeptide or a peptide encoded by a polynucleotide that hybridizes under conditions of high stringency to a polynucleotide that encodes the reference polypeptide or its complement, wherein conditions of high stringency comprise hybridization reaction at about 60° C. in about 1×SSC.

19. The isolated polynucleotide of claim 17, wherein the N-terminus and/or the C-terminus ligand comprises polypeptide (FKBP) or a biological equivalent thereof, wherein a biological equivalent of FKBP is a peptide that has at least 80% sequence identity to FKBP or a peptide encoded by a polynucleotide that hybridizes under conditions of high stringency to a polynucleotide that encodes FKBP or its complement, wherein conditions of high stringency comprise hybridization reaction at about 60° C. in about 1×SSC and optionally operatively linked to expression and/or regulatory sequences.

20. The isolated polynucleotide of claim 17, wherein N-terminus and/or the C-terminus ligand comprises cyclophilin A or a biological equivalent thereof, wherein a biological equivalent of cyclophilin A is a peptide that has at least 80% sequence identity to cyclophilin A or a peptide encoded by a polynucleotide that hybridizes under conditions of high stringency to a polynucleotide that encodes cyclophilin A or its complement, wherein conditions of high stringency comprise hybridization reaction at about 60° C. in about 1×SSC and optionally operatively linked to expression and/or regulatory sequences.

21. An isolated vector or isolated host cell comprising an isolated polynucleotide of claim 17.

22. (canceled)

23. A method for preparing an ELP-fusion, comprising expressing the polynucleotide of claim 17.

24. The method of claim 23, further comprising isolating the ELP-fusion expressed by the polynucleotide.

25. The method of claim 24, further comprising preparing a composition comprising mixing the therapeutic agent and the ELP-fusion and subsequently raising the temperature of the above the transition temperature of the ELP.

26. A method for preparing the agent of claim 1, comprising preparing a composition comprising the therapeutic agent and the ELP component and subsequently raising the temperature of the composition above the transition temperature of the ELP.
